SEQUENCE LISTING (1 sequence)

SEQ ID NO: 1 - protein, 169 aa, HUMAN IL-1ra beta
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

(19) United States
(12) Patent Application Publication
(10) Pub. No.: US 2003/0143230 A1
(43) Pub. Date: Jul. 31, 2003

(54) Combination of an IL-1/18 inhibitor with a TNF inhibitor for the treatment of inflammation

(75) Inventors: Christopher A. Gabel, Ledyard, CT (US); Mark A. Dombroski, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/306,746

(22) Filed: Nov. 27, 2002

Related U.S. Application Data
(60) Provisional application No. 60/334,419, filed on Nov. 30, 2001.

(51) Int. Cl. A61K 39/395; A61K 31/34

(52) U.S. Cl. 424/145.1; 514/471

(57) ABSTRACT

The invention relates to compositions and methods for treating or preventing inflammation, including rheumatoid arthritis (RA). The method comprises administering to mammals in need thereof an effective amount of a composition containing an agent that inhibits IL-1/18 combination with a TNF inhibitor.

### BACKGROUND OF THE INVENTION

The present invention relates generally to a combination of an Interleukin-1 (IL-1) and/or 18 (IL-18) inhibitor with a Tumor Necrosis Factor (TNF) inhibitor. Such combinations are useful pharmaceutical compositions and are useful for treating inflammation, including rheumatoid arthritis.

Inflammation is the body's defense reaction to injury such as those caused by mechanical damage, infection, or antigenic stimulation. An inflammatory reaction may be expressed pathologically when inflammation is induced by an inappropriate stimulus such as an autoantigen, expressed in an exaggerated manner, or persists well after the removal of the injurious agents. Under these conditions, inflammation may be expressed chronically. The mediation of acute inflammatory diseases such as septic shock and chronic inflammatory diseases such as rheumatoid arthritis and inflammatory bowel disease has been linked to the pro-inflammatory activities of IL-1, IL-18 and TNF.

IL-1, IL-18 and TNF are naturally occurring species that are often referred to as cytokines. Cytokines are extracellular proteins that modify the behavior of cells, particularly those cells that are in the immediate area of cytokine synthesis and release.

IL-1 is one of the most potent inflammatory cytokines yet discovered and is thought to be a key mediator in many diseases and medical conditions. IL-1, which is manufactured, though not exclusively, by cells of the macrophage/monocyte lineage, may be produced in two forms, 1L-1 alpha (IL-lα) and 1L-1 beta (IL-1β), which play a key role early in the inflammatory response (for a review see C. A. Dinarello, Blood, 87:2095-2147 (1996) and references therein). Both proteins are made as 31 kDal intracellular precursor proteins which are cleaved and secreted to yield mature carboxy-terminal 17 kDal fragments which are biologically active. In the case of IL-1β, this cleavage involves an Intracellular Cysteine Protease, known as ICE, which is required to release the active fragment from the inactive precursor. The precursor of IL-1α is active.

IL-lα and IL-1β act by binding to cell surface receptors (IL-1r) found on almost all cell types and triggering a range of responses either alone or in concert with other secreted factors. These range from effects on proliferation (e.g. of fibroblasts, T cells), apoptosis (e.g. A375 melanoma cells), cytokine induction (e.g. TNF, IL-1, IL-8), receptor activation (e.g. E-selectin), eicosanoid production (e.g. PGE2) and the secretion of degradative enzymes (e.g. collagenase). To achieve this, IL-1 activates transcription factors such as NF-κB and AP-1. Several of the activities of IL-1 action on target cells are believed to be mediated through activation of kinase cascades that have also been associated with cellular stresses, such as the stress activated MAP kinases JNK/SAPK and p38.

Soluble IL-1 receptors (IL-1sr) have been used as therapeutic agents to bind to and inactivate IL-1, such as described in U.S. Pat. Nos. 5,081,228; 5,180,812; 5,767,064; and reissue RE 35,450; and European Patent Publication EP 460,846.

A third member of the IL-1 family has also been discovered which acts as a natural antagonist of IL-1α and IL-1β by binding to the IL-1 receptor but not transducing an intracellular signal or a biological response. The protein has been called IL-1ra (for IL-1 receptor antagonist).

Therapies involving the administration of IL-1ra polypeptide have been described in various patents and publications, such as: Canadian Patent Application Nos. 2039458 and 2039458, U.S. Pat. Nos. 5,508,262, 5,880,096, 5,861,476, 5,786,331, 5,767,234, 5,608,035, WO 97/28828, WO 99/11292, WO 95/20973, WO 97/28828, and WO 98/24477.

Many studies using IL-1ra polypeptide, soluble IL-1r (derived from the intracellular domain of the type I IL-1r), antibodies to IL-1α or β, and transgenic knockouts of these genes have shown conclusively that the IL-1 family plays a key role in a number of pathophysiologies (see C. A. Dinarello, Blood 87:2095-2147 (1996) for a review). For example, IL-1ra polypeptide has been shown to be effective in animal models of septic shock, rheumatoid arthritis, graft versus host disease, stroke, cardiac ischemia, and is currently in clinical trials for some of these indications. See Ohlsson et al., 1990, “Interleukin-1 receptor antagonist reduced mortality from endotoxin shock”, Nature 348:550-551; Aiura et al., 1991, “Interleukin-1 receptor antagonist blocks hypotension in rabbit model of gram-positive septic shock”, Cytokine 4:498; Fischer et al., 1991, “A comparison between effects of interleukin-1α administration and sublethal endotoxemia in primates”, Am. J. Physiol. 261:R444; Waage and Espevik, 1988, “Interleukin-1 potentiates the lethal effect of tumor necrosis factor/cachectin in mice”, J. Exp. Med. 1678:1987; Fischer et al., “Interleukin-1 Receptor Blockade Improves Survival and Hemodynamic Performance in E. coli Septic Shock . . . ”, J. Clin. Invest. 89:1551-1557; Granowitz et al., 1992, “Pharmacokinetics, Safety, Immunomodulatory Effects of Human Recombinant Interleukin-1 Receptor Antagonist in Healthy Humans”, Cytokine 4(5):353-360; Bloedow et al., 1992, “Intravenous Disposition of Interleukin-1 Receptor Antagonist in Healthy Volunteers”, Amer. Soc. Clin. Pharm. and Therapeutics, Orlando, Fla. (Abstract). Moreover, IL-1α and β have shown some potential as hematopoietic stem cell stimulators with potential as radio- and chemo-protectants.

Human interleukin-18 (IL-18) is another member of the interleukin family that has recently been identified. IL-18 is a cytokine that is synthesized as a biologically inactive 193 amino acid precursor protein (Ushio et al., J. Immunol. 15 6:4274, 1996). Cleavage of the precursor protein, for example by caspase-1 or caspase-4, liberates the 156 amino acid mature protein (Gu et al., Science 275:206, 1997; Ghayur et al., Nature 386:619, 1997), which exhibits biological activities that include the costimulation of T cell proliferation, the enhancement of NK cell cytotoxicity, the induction of IFN-γ production by T cells and NK cells, and the potentiation of T helper type I (Th I) differentiation (Okamura et al., Nature 378:88, 1995; Ushio et al., J. Immunol. 156:4274, 1996; Micallef et al., Eur. J. Immunol. 26:1647, 1996; Kohno et al., J. Immunol. 158:1541, 1997; Zhang et al., Infect. Immunol. 65:3594, 1997; Robinson et al., Immunol 7:571, 1997). In addition, IL-18 is an efficacious inducer of human monocyte proinflammatory mediators, including IL-8, tumor necrosis factor-α, and prostaglandin E2 (PGE2) (Ushio, S. et al., J. Immunol. 156:4274-4279, 1996; Puren, A. J. et al., J. Clin. Invest. 10:711-721, 1997).

The previously cloned IL-I receptor-related protein (IL-I Rrp) (Parnet et al., J. Biol. Chem. 271:3967, 1996) has also recently been identified as a subunit of the IL-18 receptor (Kd=18 nM) (Torigoe et al., J. Biol. Chem. 272:25737, 1997). A second subunit of the IL-18 receptor exhibits homology to the IL-1 receptor accessory protein, and has been termed AcPL (for accessory protein-like). Expression of both IL-1 Rrp and AcPL are required for IL-18 induced NF-κβ and JNK activation (Born et al., J. Biol. Chem. 273:29445, 1998). In addition to NF-κβ and JNK, IL-18 signals through IL-1 receptor-associated kinase (IRAK), p561ck (LCK), and mitogen-activated protein kinase (MAPK) (Micallef et al., Eur. J. Immunol. 26:1647, 1996; Matsumoto et al., Biophys. Biochem. Res. Comm. 234:454, 1997; Tsuji-Takayama et al., Biochem. Biophys. Res. Comm. 237:126, 1997).

Th I cells, which produce proinflammatory cytokines such as IFN-7, IL-2 and TNF-α (Mosmann et al., J. Immunol. 136:2348, 1986), have been implicated in mediating many of autoimmune diseases, including multiple sclerosis (MS), rheumatoid arthritis (RA), insulin dependent diabetes (IDDM), inflammatory bowel disease (IBD), and psoriasis (Mosmann and Sad, Immunol. Today 17:138, 1996). Thus, antagonism of a Th I-promoting cytokine such as IL-18 is expected to inhibit disease development. IL-18 specific mAbs could be used as an antagonist.

Numerous additional receptors, antagonists and antibodies for IL-18 have been identified. Furthermore, soluble forms of such receptors are under investigation to determine to what extent they inhibit IL-18 activity and ameliorate any inflammatory and/or autoimmune diseases attributable to IL-18 signaling, see, for example, International Patent Publication WO 99/37772.

A series of diarylsulfonylureas (“DASUs”) have also been identified, which are potent inhibitors of stimulus-coupled post-translational processing of IL-1 and inhibitors of IL-18. These compounds are described and claimed in PCT application WO 98/32733 filed Dec. 29, 1997, which entered the United States national stage as U.S. patent application Ser. No. 09/341,782 on Aug. 16, 1999, the entire disclosure of which is hereby incorporated by reference for all purposes. Because IL-1 and IL-18 are important mediators of inflammation and inhibition of their function provides therapeutic relief in animal models of disease (Cominelli, F. et al. J. Clin. Invest. 86:972-980 (1990); Akeson, A. L. et al. J. Biol. Chem. 271:30517-30523 (1996); Caron, J. P. et al. Arthritis Rheum. 39:1535-1544 (1996); Okamura, H. et al. Nature 378:88-91 (1995); Rothwell, N. J. Clin. Invest. 100:2648-2652 (1997)), agents that disrupt the process of stimulus-coupled post-translational processing will be useful for the treatment in men and animals of disorders that are sustained by inflammatory mediators. These include rheumatoid arthritis, osteoarthritis, asthma, inflammatory bowel disease, ulcerative colitis, neurodegeneration, atherosclerosis, and psoriasis.

TNF's are a separate class of cytokines produced by numerous cell-types, including monocytes and macrophages. At least two TNF's have been previously described, specifically TNF alpha (TNF-α) and TNF beta (TNF-β or lymphotoxin).

In unstimulated cells, TNF-α is bound in the cell. TNF-α Converting Enzyme (TACE) is responsible for cleavage of cell bound TNF-α. TNF-α is recognized to be involved in many infectious and autoimmune diseases (W. Friers, FEBS Letters, 285, 199 (1991)). Furthermore, it has been shown that TNF-α is the prime mediator of the inflammatory response seen in sepsis and septic shock (Spooner, et al., Clinical Immunology and Immunopathology, 62 S11 (1992)). There are two forms of TNF-α, a type II membrane protein of relative molecular mass 26,000 (26 kD) and a soluble 17 kD form generated from the cell bound protein by specific proteolytic cleavage. The soluble 17 kD form of TNF-α is released by the cell and is associated with the deleterious effects of TNF-α. This form of TNF-α is also capable of acting at sites distant from the site of synthesis. Thus, inhibitors of TACE prevent the formation of soluble TNF-α and prevent the deleterious effects of the soluble factor (see U.S. Pat. No. 5,830,742 issued Nov. 3, 1998, U.S. Pat. No. 5,594,106 issued Jan. 14, 1997 and International Patent Publication WO 97/35538 published Oct. 2, 1997).

Soluble TNF receptors (TNFsr) have demonstrated effectiveness at ameliorating inflammation, see for example etanercept (Enbrel). Etanercept is described in U.S. Pat. Nos. 5,395760, 5,712,155, 5,945,397, 5,344,915, and reissue RE 36,755.

Antibodies for TNF or TNFr are known to be useful in the treatment of inflammation and include infliximab (Remicade®), CDP-870 and adalimumab (D2E7). Infliximab is described in U.S. Pat. Nos. 5,698,195 and 5,656,272. Adalimumab is described in International Patent Publication WO 97/29131. Methods of producing humanized antibodies such as CDP-870 are described in European Patent Publications 120,694, 460,167 and 516,785.

U.S. Provisional Patent Application entitled “Selective Inhibitors of Aggrecanase in Osteoarthritis Treatment,” filed Aug. 12, 1999 refers to certain small molecule TACE inhibitors and to additional methods of preparing hydroxamic acids. U.S. Non-Provisional Application entitled “TACE Inhibitors,” filed Aug. 12, 1999, refers to heterocyclic hydroxamic acids. Each of the above referenced publications and applications is hereby incorporated by reference in its entirety.

WO 93/21946 describes combination therapies for conditions that are mediated by IL-1 or TNF. The therapies use IL-1 inhibitors, especially IL-1ra, in combination with a 30 KDa TNF inhibitor. However, no combination of IL-1 processing and release inhibitor, IL-18 inhibitor or TACE inhibitors was described.

It has now been discovered that the present combination of an agent that inhibits the propagation of IL-1/18 with a TNF inhibitor (preferably a TACE inhibitor) provides a synergistic benefit over the individual agents, alone.

### SUMMARY OF THE INVENTION

The invention provides for compositions comprising an amount of an IL-1 and/or 18 inhibitor in combination with an amount of a Tumor Necrosis Factor (TNF) inhibitor, wherein the amount of the two components is effective for treating inflammation and a pharmaceutically acceptable carrier. This invention also provides for methods of treatment comprising administering such combinations.

A specific embodiment of the above referenced composition and method combinations are those combinations wherein an amount of an IL-1 inhibitor is combined with an amount of a Tumor Necrosis Factor (TNF) inhibitor, wherein the amount of the two components is effective for treating inflammation and a pharmaceutically acceptable carrier.

Another specific embodiment of the above referenced composition and method combinations are those combinations wherein an amount of an IL-18 inhibitor is combined with an amount of a Tumor Necrosis Factor (TNF) inhibitor, wherein the amount of the two components is effective for treating inflammation and a pharmaceutically acceptable carrier.

Another specific embodiment of the above referenced composition and method combinations are those combinations wherein an amount of an IL-1 inhibitor and an IL-18 inhibitor are combined with an amount of a Tumor Necrosis Factor (TNF) inhibitor, wherein the amount of the three components is effective for treating inflammation and a pharmaceutically acceptable carrier.

Another specific embodiment of the above referenced composition and method combinations are those combinations wherein an amount of a dual IL-1 and IL-18 inhibitor is combined with an amount of a Tumor Necrosis Factor (TNF) inhibitor, wherein the amount of the two components is effective for treating inflammation and a pharmaceutically acceptable carrier.

Another specific embodiment of the above referenced composition and method combinations are those combinations wherein said IL-1 inhibitor is an IL-1ra (preferably anakinra).

Another specific embodiment of the above referenced composition and method combinations are those combinations wherein said IL-1/18 inhibitor is selected from the group consisting of IL-1 processing and release inhibitors.

Another specific embodiment of the above referenced composition and method combinations are those combinations wherein said IL-1/18 inhibitor is a soluble IL-1r or IL-18r (IL-1sr or IL-18 sr) or an antibody to IL-1, IL-1r, IL-18or IL-18r.

IL-1 processing and release inhibiting agents are selected from the group consisting of inhibitors of ICE, inhibitors of caspase, and inhibitors of IL-1 post-translational processing. More preferably, the IL-1 processing and release inhibiting agent is an inhibitor of IL-1 post-translational processing. Particularly preferred inhibitors of IL-1 post-translational processing are inhibitors of IL-1 stimulus-coupled post-translational processing, and more particularly, .anion transport inhibitors, and diuretics such as thiazides and ethacrynic acid. A particularly preferred diuretic is ethacrynic acid.

Another specific embodiment of the above referenced composition and method combinations are those combinations wherein said IL-1 inhibitor is an IL-1 processing and release inhibitor selected from the group consisting of an ICE inhibitor, a caspase inhibitor, and an IL-1 post-translational processing inhibitor.

Another specific embodiment of the above referenced composition and method combinations are those combinations wherein said IL-1 inhibitor is an ICE inhibitor.

Another specific embodiment of the above referenced composition and method combinations are those combinations wherein said IL-1 inhibitor is a caspase inhibitor.

A specific embodiment of the above referenced composition and method combinations are those combinations wherein said IL-1 inhibitor is an IL-1 post-translational processing inhibitor.

A specific embodiment of the above referenced composition and method combinations are those combinations wherein said IL-1 inhibitor is an IL-1 post-translational processing inhibitor selected from diarylsulfonylureas.

IL-1 processing and release inhibiting agents that are preferred are those that have IC₅₀ values of less than 50 μM, more preferably less than 1 μM, and most preferably less than 100 nM (as determined in one of the in vitro assays described herein).

A particularly preferred class of IL-1 processing and release inhibiting agents that are useful in the methods and compositions of the present invention are diarylsulfonylureas. Preferred diarylsulfonylureas are compounds of formula I

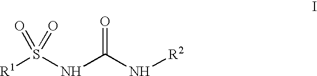

or a pharmaceutically acceptable salt thereof, wherein R¹ and R² are each independently a group of formula II

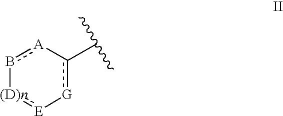

wherein the broken lines (- - -) represent optional double bonds;

n is 0, 1, 2 or 3;

A, B, D, E and G are each independently oxygen, sulfur, nitrogen or CR⁵R⁶ wherein R⁵ and R⁶ are each independently selected from (1) hydrogen, (2) (C₁-C₆)alkyl optionally substituted by one or two groups selected from (C₁-C₆)alkylamino, (C₁-C₆)alkylthio, (C₁-C₆)alkoxy, hydroxy, cyano, perfluoro(C₁-C₆)alkyl, (C₆-C₁₀)aryl, (C₅-C₉)heteroaryl, (C₆-C₁₀)arylamino, (C₆-C₁₀)arylthio, (C₆-C₁₀)aryloxy wherein the aryl group is optionally substituted by (C₁-C₆)alkoxy, (C₁-C₆)acyl, carboxy, hydroxy or halo; (C₅-C₉)heteroarylamino, (C₅-C₉)heteroarylthio, (C₅-C₉)heteroaryloxy, (C₆-C₁₀)aryl(C₆-C₁₀)aryl, (C₃-C₆)cycloalkyl, hydroxy, piperazinyl, (C₆-C₁₀)aryl(C₁-C₆)alkoxy, (C₅-C₉)heteroaryl(C₁-C₆)alkoxy, (C₁-C₆)acylamino, (C₁-C₆)acylthio, (C₁-C₆)acyloxy, (C₁-C₆)alkylsulfinyl, (C₆-C₁₀)arylsulfinyl, (C₁-C₆)alkylsulfonyl, (C₆-C₁₀)arylsulfonyl, amino, (C₁-C₆)alkylamino or ((C₁-C₆)alkyl)2amino; (3) halo, (4) cyano, (5) amino, (6) hydroxy, (7) perfluoro(C₁-C₆)alkyl, (8) perfluoro(C₁-C₆)alkoxy, (9) (C₂-C₆)alkenyl, (10) carboxy(C₂-C₆)alkenyl, (11) (C₂-C₆)alkynyl, (12) (C₁-C₆)alkylamino, (13) ((C₁-C₆)alkyl)2amino, (14) (C₁-C₆)alkylsulfonylamido, (15) (C₁-C₆)alkylsulfinyl, (16) (C₁-C₆)alkylsulfonyl, (17) aminosulfonyl, (18) (C₁-C₆)alkylaminosulfonyl, (19) ((C₁-C₆)alkyl)2aminosulfonyl, (20) (C₁-C₆)alkylthio, (21) (C₁-C₆)alkoxy, (22) perfluoro(C₁-C₆)alkyl, (23) (C₆-C₁₀)aryl, (24) (C₅-C₉)heteroaryl, (25) (C₆-C₁₀)arylamino, (26) (C₆-C₁₀)arylthio, (27) (C₆-C₁₀)aryl(C₁-C₆)alkoxy, (28) (C₅-C₉)heteroarylamino, (29) (C₅-C₉)heteroarylthio, (30) (C₅-C₉)heteroaryloxy, (31) (C₃-C₆)cycloalkyl, (32) (C₁-C₆)alkyl(hydroxymethylene), (33) piperidyl, (34) pyridinyl, (35) thienyl, (36) furanyl, (37) (C₁-C₆)alkylpiperidyl, (38) (C₁-C₆)acylamino, (39) (C₁-C₆)acylthio, (40) (C₁-C₆)acyloxy, (41) R⁷(C₁-C₆)alkyl wherein R⁷ is (C₁-C₆)acylpiperazino, (C₆-C₁₀)arylpiperazino, (C₅-C₉)heteroarylpiperazino, (C₁-C₆)alkylpiperazino, (C₆-C₁₀)aryl(C₁-C₆)alkylpiperazino, (C₅-C₉)heteroaryl(C₁-C₆)alkylpiperazino, morpholino, thiomorpholino, piperidino, pyrrolidino, piperidyl, (C₁-C₆)alkylpiperidyl, (C₆-C₁₀)arylpiperidyl, (C₅-C₉)heteroarylpiperidyl, (C₁-C₆)alkylpiperidyl(C₁-C₆)alkyl, (C₆-C₁₀)arylpiperidyl(C₁-C₆)alkyl, (C₅-C₉)heteroarylpiperidyl(C₁-C₆)alkyl or (C₁-C₆)acylpiperidyl;

(42) or a group of formula III

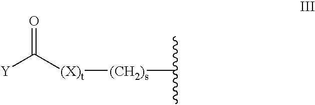

wherein s is 0 to 6;

t is 0 or 1;

X is oxygen or NR⁸ wherein R⁸ is hydrogen, (C₁-C₆)alkyl or (C₃-C₇)cycloalkyl(C₁-C₆)alkyl;

Y is hydrogen, hydroxy, (C₁-C₆)alkyl optionally substituted by halo, hydroxy or cyano; (C₁-C₆)alkoxy, cyano, (C₂-C₆)alkynyl, (C₆-C₁₀)aryl wherein the aryl group is optionally substituted by halo, hydroxy, carboxy, (C₁-C₆)alkyl, (C₁-C₆)alkoxy, perfluoro(C₁-C₆)alkyl, (C₁-C₆)alkoxy(C₁-C₆)alkyl or NR⁹R¹⁰; wherein R⁹ and R¹⁰ are each independently selected from the group consisting of hydrogen and (C₁-C₆)alkyl optionally substituted by (C₁-C₆)alkylpiperidyl, (C₆-C₁₀)arylpiperidyl, (C₅-C₉)heteroarylpiperidyl, (C₆-C₁₀)aryl, (C₅-C₉)heteroaryl or (C₃-C₆)cycloalkyl; piperidyl, (C₁-C₆)alkylpiperidyl, (C₆-C₁₀)arylpiperidyl, (C₅-C₉)heteroarylpiperidyl, (C₁-C₆)acylpiperidyl, (C₆-C₁₀)aryl, (C₅-C₉)heteroaryl, (C₃-C₆)cycloalkyl, R¹¹(C₂-C₆)alkyl, (C₁-C₅)alkyl(CHR¹¹)(C₁-C₆)alkyl wherein R¹¹ is hydroxy, (C₁-C₆)acyloxy, (C₁-C₆)alkoxy, piperazino, (C₁-C₆)acylamino, (C₁-C₆)alkylthio, (C₆-C₁₀)arylthio, (C₁-C₆)alkylsulfinyl, (C₆-C₁₀)arylsulfinyl, (C₁-C₆)alkylsulfoxyl, (C₆-C₁₀)arylsulfoxyl, amino, (C₁-C₆)alkylamino, ((C₁-C₆)alkyl)₂amino, (C₁-C₆)acylpiperazino, (C₁-C₆)alkylpiperazino, (C₆-C₁₀)aryl(C₁-C₆)alkylpiperazino, (C₅-C₉)heteroaryl(C₁-C₆)alkylpiperazino, morpholino, thiomorpholino, piperidino or pyrrolidino; R¹²(C₁-C₆)alkyl, (C₁-C₅)alkyl(CHR¹²)(C₁-C₆)alkyl wherein R¹² is piperidyl or (C₁-C₆)alkylpiperidyl; and CH(R¹³)COR¹⁴ wherein R¹⁴ is as defined below and R¹³ is hydrogen, (C₁-C₆)alkyl, (C₆-C₁₀)aryl(C₁-C₆)alkyl, (C₅-C₉)heteroaryl(C₁-C₆)alkyl, (C₁-C₆)alkylthio(C₁-C₆)alkyl, (C₆-C₁₀)arylthio(C₁-C₆)alkyl, (C₁-C₆)alkylsulfinyl(C₁-C₆)alkyl, (C₆-C₁₀)arylsulfinyl(C₁-C₆)alkyl, (C₁-C₆)alkylsulfonyl(C₁-C₆)alkyl, (C₆-C₁₀)arylsulfonyl(C₁-C₆)alkyl, hydroxy(C₁-C₆)alkyl, amino(C₁-C₆)alkyl, (C₁-C₆)alkylamino(C₁-C₆)alkyl, (C₁-C₆)alkylamino)₂(C₁-C₆)alkyl, R¹⁵R¹⁶NNCO(C₁-C₆)alkyl or R¹⁵OCO(C₁-C₆)alkyl wherein R¹⁵ and R¹⁶ are each independently selected from the group consisting of hydrogen, (C₁-C₆)alkyl, (C₆-C₁₀)aryl(C₁-C₆)alkyl and (C₅-C₉)heteroaryl(C₁-C₆)alkyl; and R¹⁴ is R 17O or R¹⁷R¹⁸N wherein R¹⁷ and R¹⁸ are each independently selected from the group consisting of hydrogen, (C₁-C₆)alkyl, (C₆-C₁₀)aryl(C₁-C₆)alkyl and (C₅-C₉)heteroaryl(C₁-C₆)alkyl;

(43) or a group of formula IV

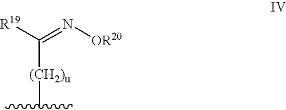

wherein u is 0, 1 or 2;

R¹⁹ is hydrogen, (C₁-C₆)alkyl or perfluoro(C₁-C₆)alkyl;

R²⁰ is hydrogen, (C₁-C₆)alkyl, (C₁-C₆)carboxyalkyl or (C₆-C₁₀)aryl(C₁-C₆)alkyl.

(44) or a group of formula V

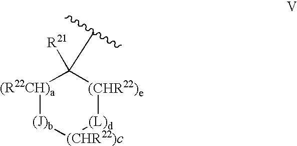

wherein a is 0, 1 or 2;

b is 0 or 1;

c is 1, 2 or 3;

d is 0 or 1;

e is 0, 1 or 2;

J and L are each independently oxygen or sulfur;

R²¹ is hydrogen, hydroxy, fluoro, (C₁-C₆)alkyl, (C₁-C₆)alkoxy, halo(C₁-C₆)alkyl, amino, (C₁-C₆)acylamino or NR²⁶R²⁷ wherein R²⁶ and R²⁷ are each independently selected from hydrogen, (C₁-C₆)alkyl or (C₆-C₁₀)aryl; and

R²² is hydrogen, (C₁-C₆)alkyl optionally substituted by hydroxy, halo, (C₁-C₆)alkylthio, (C₁-C₆)alkylsulfinyl or (C₁-C₆)alkylsulfonyl;

or in formula II when n is 1 and B and D are both CR⁵, the two R⁵ groups may be taken together with the carbons to which they are attached to form a group of formula VI

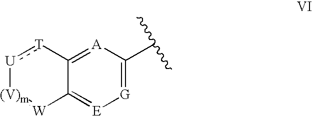

wherein the broken lines represent optional double bonds;

m is 0 or 1; and

T, U, V and W are each independently oxygen, sulfur, CO, nitrogen or CR⁵R⁶ wherein R⁵ and R⁶ are as defined above;

or when A and B are both CR⁵, or when n is 1 and B and D are both CR⁵, or when D and E are both CR⁵, or when E and G are both CR⁵, the two R⁵ groups may be taken together with the adjacent carbons to which they are attached to form a (C₅-C₆)cycloalkyl group optionally substituted by hydroxy or a benzo group.

An embodiment of the compounds of formula I (above) requires that R² must be aromatic.

Another embodiment of the composition and method combinations is that group of combinations wherein said IL-1 inhibiting component is a compound of formula I (above) wherein the groups of formulae II and VI do not have two oxygens, two sulfurs or an oxygen and sulfur defined in adjacent positions.

More preferred diarylsulfonylureas useful for the methods and compositions of the present invention are compounds of formula I wherein R¹ is a group of formula II

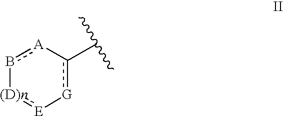

wherein the broken lines represent optional double bonds;

n is 0;

A is CR⁵ wherein R⁵ is hydrogen or halo;

B and E are both independently CR⁵ wherein R⁵ is (1) hydrogen, (2) cyano, (3) halo, (4) (C₁-C₆)alkyl optionally substituted by one or two hydroxy; (5) (C₃-C₇)cycloalkylaminosulfonyl, (6) (C₁-C₆)alkylaminosulfonyl, or (7) a group of formula III

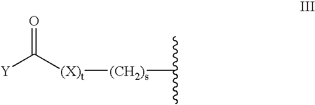

wherein s is 0;

t is 0 ; and

Y is hydrogen, (C₁-C₆)alkyl optionally substituted by halo; or (C₁-C₆)alkoxy(C₁-C₆)alkyl;

D is absent;

G is oxygen, sulfur or CR⁵ wherein R⁵ is hydrogen or halo.

More preferred diarylsulfonylureas useful for the methods and compositions of the present invention are compounds of formula I wherein said R² is a group of formula II

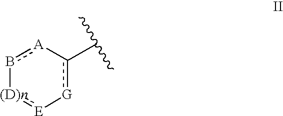

wherein the broken lines represent double bonds;

n is 1;

A is CR⁵ wherein R⁵ is halo or (C₁-C₆)alkyl;

B is CR⁵ wherein R⁵ is hydrogen or halo;

D is CR⁵ wherein R⁵ is hydrogen, halo, cyano or a group of formula III

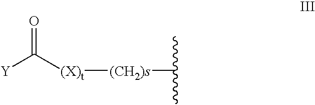

wherein s is 0;

t is 0; and

Y is NH₂;

E is CR⁵ wherein R⁵ is hydrogen or halo; and

G is CR⁵ wherein R⁵ is halo or (C₁-C₆)alkyl.

More preferred diarylsulfonylureas useful for the methods and compositions of the present invention are compounds of formula I wherein said R² is a group of formula II

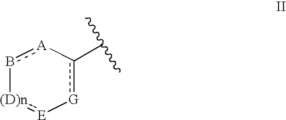

wherein the broken lines represent double bonds;

is 1; and A, B E and G, are each CR⁵, and the two advent R⁵ groups of A and B and E and G are taken together with the adjacent carbons to which they are attached form a (C₅-C₆)cycloalkyl group.

More preferred diarylsulfonylureas useful for the methods and compositions of the present invention are compounds of formula I wherein said R²is a group of formula

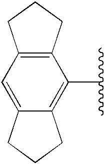

Particular species of diarylsulfonylureas that are useful in the compositions and methods of the present invention may be selected from the group consisting of

1-(1,2,3,5,6,7-Hexahydro-s-indacen-4-yl)-3-[4-(1-hydroxy-1-methyl-ethyl)-furan-2-sulfonyl]-urea;

1-(2,6-Diisopropyl-phenyl)-3-[4-(1-hydroxy-1-methyl-ethyl)-furan-2-sulfonyl]-urea;

1-(1,2,3,5,6,7-Hexahydro-4-aza-s-indacen-8-yl)-3-[4-(1-hydroxy-1-methyl-ethyl)-furan-2-sulfonyl]-urea;

1-(4-Chloro-2,6-diisopropyl-phenyl)-3-[3-(1-hydroxy-1-methyl-ethyl)-benzenesulfonyl]-urea;

1-(1,2,3,5,6,7-Hexahydro-s-indacen-4-yl)-3-[4-(1-hydroxy-1-methyl-ethyl)-thiophene-2-sulfonyl]-urea;

1-(4-[1,3]Dioxolan-2-yl-furan-2-sulfonyl)-3-(1,2,3,5,6,7-hexahydro-s-indacen-4-yl)-urea;

1-(2,6-Diisopropyl-phenyl)-3-[4-(1-hydroxy-1-methyl-ethyl)-thiophene-2-sulfonyl]-urea;

1-(4-Acetyl-thiophene-2-sulfonyl)-3-(1,2,3,5,6,7-hexahydro-s-indacen-4-yl)-urea;

1-(1H-Benzoimidazole-5-sulfonyl)-3-(1,2,3,5,6,7-hexahydro-s-indacen-4-yl)-urea;

1-(1,2,3,5,6,7-Hexahydro-s-indacen-4-yl)-3-[4-(1-hydroxy-1-methyl-ethyl)-thiophene-2-sulfonyl]-urea;

1-(8-Chloro-1,2,3,5,6,7-hexahydro-s-indacen-4-yl)-3-[4-(1-hydroxy-1-methyl-ethyl)-furan-2-sulfonyl]-urea;

1-(4-Acetyl-furan-2-sulfonyl)-3-(1,2,3,5,6,7-hexahydro-s-indacen-4-yl)-urea;

1(8Fluoro-1,2,3,5,6,7-hexahydro-s-indacen-4-yl)-3-[4-(1-hydroxy-1methyl-ethyl)-furan-2-sulfonyl]-urea;

1-(4-Fluoro-2,6-diisopropyl-phenyl)-3-[3-(1-hydroxy-1-methyl-ethyl)-benzenesulfonyl]-urea;

1-(6-Fluoro-1H-benzoimidazole-5-sulfonyl)-3-(1,2,3,5,6,7-hexahydro-s-indacen-4-yl)-urea;

1-(4-Chloro-2,6-diisopropyl-phenyl)-3-(1H-indole-6-sulfonyl)-urea;

1-(4-Chloro-2,6-diisopropyl-phenyl)-3-(5-fluoro-1H-indole-6-sulfonyl)-urea;

1-(1,2,3,5,6,7-Hexahydro-s-indacen-u-yl)-3-(1H-indole-6-sulfonyl)-urea;

1-(5-Fluoro-1H-indole-6-sulfonyl)-3-(1,2,3,5,6,7-hexanhydro-5-indacen-4-yl)-urea;

1-[4-Chloro-2,6-diisopropyl-phenyl]-3-[2-fluoro-5-(2-methyl-(1,3)dioxolan-2-yl)-benzenesulfonyl]-urea;

3-[3-[4-Chloro-2,6-diisopropyl-phenyl]-ureidosulfonyl]-N-methyl-benzenesulfonamide;

1-[2-Fluoro-5-(2-methyl-(1,3)dioxolan-2-yl)benzenesulfonyl]-3-1,2,3,5,6,7-hexahydro-indacen-4-yl)-urea;

1-(4-Chloro-2,6-diisopropyl-phenyl)-3-[2-fluoro-5-oxiranylbenzenesulfonyl]-urea;

1-(1,2,3,5,6,7-Hexahydro-s-indacen-4-yl)-3-[2-fluoro-5-oxiranylbenzenesulfonyl]-urea; and

3-[3-(1,2,3,5,6,7-Hexahydro-S-indacen-4-yl)-ureidosulfonyl]-N-methyl-benzenesulfonamide.

Particularly preferred species among those diarylsulfonylureas useful in the compositions of the present invention are

1-(1,2,3,5,6,7-Hexahydro-s-indacen-4-yl)-3-[4-(1-hydroxy-1-methyl-ethyl)-furan-2-sulfonyl]-urea;

1-(2,6-Diisopropyl-phenyl)-3-[4-(1-hydroxy-1-methyl-ethyl )-furan-2-sulfonyl]-urea;

4-Chloro-2,6-diisopropyl-phenyl-3-[4-(1-hydroxy-1-methyl-ethyl)-furan-2-sulfonyl]-urea;

1,2,3,5,6,7-Hexahydro-4-aza-s-indacen-8-yl-3-[4-(1-hydroxy-1-methyl-ethyl )-furan-2-sulfonyl]-urea;

8-Chloro-1,2,3,5,6,7-hexahydro-s-indacen-4-yl-3-[4-(1-hydroxy-1-methyl-ethyl)-furan-2-sulfonyl]-urea;

8-Fluoro-1,2,3,5,6,7-hexahydro-s-indacen-4-yl-3-[4-(1-hydroxy-1-methyl-ethyl)-furan-2-sulfonyl]-urea; and

4-Fluoro-2,6-diisopropyl-phenyl-3-[4-(1-hydroxy-1-methyl-ethyl)-furan-2-sulfonyl]-urea.

Another class of IL-1 processing and release inhibitors useful in the compositions of the present invention are inhibitors of ICE. In particular, preferred inhibitors of ICE are compounds and pharmaceutically acceptable salts thereof selected from the group consisting of ICE inhibitor compounds of U.S. Pat. Nos. 5,656,627, 5,847,135, 5,756,466, 5,716,929 and 5,874,424.

A preferred ICE inhibitor useful in the composition and method combinations of the present invention is Vertex VX740 (pralnacasan, HMR-3480), whose synthesis and activity are described in detail in U.S. Pat. No. 5,874,424.

Another embodiment of the invention of composition and method combinations is that group of combinations wherein one of the active ingredients of said combination is a soluble TNF receptor (TNFsr), an antibody for TNF or TNFr, or a TACE inhibitor.

Another embodiment of the invention of composition and method combinations is that group of combinations wherein one of the active ingredients of said combination is the Tumor Necrosis Factor (TNF) inhibitor etanercept.

Another embodiment of the invention is that group of composition and method combinations wherein one of the active ingredients of said combination is the Tumor Necrosis Factor (TNF) inhibitor infliximab.

Another embodiment of the invention is that group of composition and method combinations wherein one of the active ingredients of said combination is the Tumor Necrosis Factor (TNF) inhibitor CDP-870.

Another embodiment of the invention is that group of composition and method combinations wherein one of the active ingredients of said combination is the Tumor Necrosis Factor (TNF) inhibitor adalimumab.

Another embodiment of the invention is that group of composition and method combinations wherein one of the active ingredients of said combination is a Tumor Necrosis Factor (TNF) inhibitor selected from the group consisting of TACE inhibitors. TACE and Inhibitors thereof are described in U.S. Pat. No. 5,830,742 issued Nov. 3, 1998, U.S. Pat. No. 5,594,106 issued Jan. 14, 1997 and International Patent Publication WO 97/35538 published Oct. 2, 1997.

The present inventors have also discovered that it is possible to combine inhibitors with differential metalloprotease and reprolysin activity (preferably TACE inhibitory activity over MMP and Aggrecanase activity) with an agent that inhibits the propagation of Interleukin-1/18 (IL-1/18). One group of preferred combinations include inhibitors which selectively inhibit TACE preferentially over MMP-1. Another group of preferred combinations include inhibitors which selectively inhibit TACE and matrix metalloprotease-13 (MMP-13) preferentially over MMP-1. Another group of preferred combinations include inhibitors which selectively inhibit Aggrecanase and TACE preferentially over MMP-1. Another group of preferred combinations include inhibitors which selectively inhibit Aggrecanase, TACE and MMP-13 preferentially over MMP-1. Another group of preferred combinations include inhibitors which selectively inhibit TACE preferentially over MMP-1, Aggrecanase and MMP-13.

Another embodiment of the invention is that group of composition and method combinations wherein one of the active ingredients of said combination is a Tumor Necrosis Factor (TNF) inhibitor selected from the group of ADAM-17 (TACE) inhibitors 100 fold selective for ADAM-17 over each of MMP-1, 2, 3, 4, 5, 6, 7, 8, 9, 10, 11, 12 and 14 as each are defined in in vitro assays.

Another embodiment of the invention is that group of composition and method combinations wherein one of the active ingredients of said combination is a Tumor Necrosis Factor (TNF) inhibitor selected from the group consisting of a TACE inhibitor and the other active ingredient is an IL-1ra, preferably anakinra.

Another embodiment of the invention is that group of composition and method combinations wherein one of the active ingredients of said combination is a TACE inhibitor selected from the group consisting of an arylsulfonyl hydroxamic acid derivative.

Another embodiment of the invention is that group of composition and method combinations wherein one of the active ingredients of said combination is a arylsulfonyl hydroxamic acid derivative TACE inhibitor wherein said arylsulfonyl hydroxamic acid derivative has the formula of:

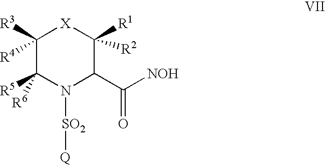

or the pharmaceutically acceptable salt thereof, wherein

X is oxygen, sulfur, SO, SO₂ or NR⁷;

R¹, R², R³, R⁴, R⁵ and R⁶ are selected from the group consisting of hydrogen, hydroxy, NH₂, —CN, (C₁-C₆)alkyl, (C₂-C₆)alkenyl, (C₆-C₁₀)aryl(C₂-C₆)alkenyl, (C₂-C₉)heteroaryl(C₂-C₆)alkenyl, (C₂-C₆)alkynyl, (C₆-C₁₀)aryl(C₂-C₆)alkynyl, (C₂-C₉)heteroaryl(C₂-C₆)alkynyl, (C₁-C₆)alkylamino, [(C₁-C₆)alkyl]₂amino, (C₁-C₆)alkylthio, (C₁-C₆)alkoxy, perfluoro(C₁-C₆)alkyl, perfluoro(C₁-C₆)alkoxy, (C₆-C₁₀)aryl, (C₂-C₉)heteroaryl, (C₆-C₁₀)arylamino, (C₆-C₁₀)arylthio, (C₆-C₁₀)aryloxy, (C₂-C₉)heteroarylamino, (C₂-C₉)heteroarylthio, (C₂-C₉)heteroaryloxy, (C₃-C₆)cycloalkyl, (C₁-C₆)alkyl(hydroxymethylene), piperidyl, (C₁-C₆)alkylpiperidyl, (C₁-C₆)acyl, (C₁-C₆)acylamino, (C₁-C₆)acylthio, (C₁-C₆)acyloxy, (C₁-C₆)alkoxy-(C═O)—, —CO₂H, H₂N—(C═O)—, (C₁-C₆)alkyl-NH—(C═O)—, and [(C₁-C₆)alky]₂—N—(C═O)—;

wherein said (C₁-C₆)alkyl is optionally substituted by one or two groups selected from (C₁-C₆)ailkylthio, (C₁-C₆)alkoxy, trifluorofmethyl, halo, —CN, (C₆-C₁₀)aryl, (C₂-C₉)heteroaryl, (C₆-C₁₀)arylamino, (C₆-C₁₀)arylthio, (C₆-C₁₀)aryloxy, (C₂-C₉)heteroarylamino, (C₂-C₉)heteroarylthio, (C₂-C₉)heteroaryloxy, (C₆-C₁₀)aryl(C₆-C₁₀)aryl, (C₃-C₆)cycloalkyl, hydroxy, piperazinyl, (C₆-C₁₀)aryl(C₁-C₆)alkoxy, (C₂-C₉)heteroaryl(C₁-C₆)alkoxy, (C₁-C₆)acylamino, (C₁-C₆)acylthio, (C₁-C₆)acyloxy, (C₁-C₆)alkylsulfinyl, (C₆-C₁₀)arylsulfinyl, (C₁-C₆)alkylsulfonyl, (C₆-C₁₀)arylsulfonyl, amino, (C₁-C₆)alkylamino or ((C₁-C₆)alkyl)₂amino;

R⁷ is hydrogen; (C₁-C₆)alkyl optionally substituted by one or more of hydroxy, —CN, (C₁-C₆)alkylamino, (C₁-C₆)alkylthio, (C₁-C₆)alkoxy, perfluoro(C₁-C₆)alkyl, (C₆-C₁₀)aryl, (C₆-C₁₀)arylthio, (C₆-C₁₀)aryloxy, (C₂-C₉)heteroarylamino, (C₃-C₆)cycloalkyl, (C₁-C₆)alkyl(hydroxymethylene), piperidyl, (C₁-C₆)alkylpiperidyl, (C₁-C₆)acyl, (C₁-C₆)acylamino, (C₁-C₆)acyloxy, (C₁-C₆)alkoxy—(C═32 O)—, —CO₂H, (C₁-C₆)alkyl—NH—(C═O)—, and [(C₁-C₆)alky]₂—N—(C═O)—; (C₆-C₁₀)arylsulfonyl; (C₁-C₆)alkylsulfonyl; (C₁-C₆)alkyl—NH—(C═O)—; (C₁-C₁₀)alkoxy-(C═O)—; (C₁-C₆)alkyl-(C═O)—; [(C₁-C₆)alky]₂—N—(C═O)—; or (R⁸R⁹N)—(C═O) where R⁸ and R⁹ are taken together with the nitrogen that they are attached to form a ring selected from azetidinyl, pyrrolidinyl, piperidinyl, morpholinyl and thiomorphonyl;

Q is (C₆-C₁₀)aryl(C₁-C₆)alkoxy(C₆-C₁₀)aryl, (C₆-C₁₀)aryl(C₁-C₆)alkoxy(C₂-C₉)heteroaryl, (C₂-C₉)heteroaryl(C₁-C₆)alkoxy(C₆-C₁₀)aryl, or (C₂-C₉)heteroaryl(C₁-C₆)alkoxy(C₂-C₉)heteroaryl, wherein each of said (C₆-C₁₀)aryl or (C₂-C₉)heteroaryl groups may optionally be substituted by one or more substituents, preferably one to three substituents per ring, most preferably one to three substituents on the terminal ring independently selected from the group consisting of halo, —CN, (C₁-C₆)alkyl optionally substituted with one or more fluorine atoms, hydroxy, hydroxy-(C₁-C₆)alkyl, (C₁-C₆)alkoxy optionally substituted with one or more fluorine atoms, (C₁-C₆)alkoxy(C₁-C₆)alkyl, HO—(C═O)—, (C₁-C₆)alkyl—O—(C═O)—, HO—(C═O)—(C₁-C₆)alkyl, (C₁-C₆)alkyl—O—(C═O)—(C₁-C₆)alkyl, (C₁-C₆)alkyl—(C═O)—O—, (C₁-C₆)alkyl—(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, (C₁-C₆)alkyl-SO₂—[N—(C₁-C₆)alkyl]—, H₂N-SO₂—, H₂N—SO₂—(C₁-C₆)alkyl, (C₁-C₆)alkyl H N—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenyl(C₁-C₆)alkyl, (C₃-C₁₀)cycloalkyl, (C₂-C₉)heterocycloalkyl, and (C₂-C₉)heteroaryl;

with the proviso that when X is SO or SO₂, and R³ and R⁴ are a substituent comprising a heteroatom, the heteroatom cannot be bonded to the ring.

Another embodiment of the invention is that group of composition and method combinations wherein one of the active ingredients of said combination is an arylsulfonyl hydroxamic acid derivative TACE inhibitor compound selected from the group consisting of:

(2S,3S)-4-[4-(3,5-difluro-benzyloxy)-benzenesulfonyl]-2-methyl-thiomorpholine-3-carboxylic acid hydroxyamide;

(2S,3S)-4-[4-(4-fluoro-benzyloxy)-benzenesulfonyl]-2-methyl-thiomorpholine-3-carboxylic acid hydroxyamide;

(2S,3R,6S)-2,6-dimethyl-4-[4-(2-methyl-benzyloxy)-benzenesulfonyl]-morpholine-3-carboxylic acid hydroxyamide;

4-(4-benzyloxy-benzenesulfonyl)-2-methyl-morpholine-3-carboxylic acid hydroxyamide;

(2S,3R,6S)-4-[4-(4-fluoro-benzyloxy)-benzenesulfonyl]-2,6-dimethyl-morpholine-3-carboxylic acid hydroxyamide;

(3R,6S)-4-[4-(4-fluoro-benzyloxy)-benzenesulfonyl]-2,2,6-trimethyl-morpholine-3-carboxylic acid hydroxyamide;

(2S,3R,6S)-6-ethyl-4-[4-(4-fluoro-benzyloxy)-benzenesulfonyl]-2-methyl-morpholine-3-carboxylic acid hydroxyamide;

(2R,3R,6S)-4-[4-(4-fluoro-benzyloxy)-benzenesulfonyl]-2,6-dimethyl-morpholine-3-10 carboxylic acid hydroxyamide;

(2R,3R,6R)-4-[4-(4-fluoro-benzyloxy)-benzenesulfonyl]-2,6-dimethyl-morpholine-3-carboxylic acid hydroxyamide;

(2S,3R,6S)-2,6-dimethyl-4-[4-(pyridin-4-ylmethoxy)-benzenesulfonyl]-morpholine-3-carboxylic acid hydroxyamide;

(2S,3R,6S)-2,6-dimethyl-4-[4-(pyridin-2-ylmethoxy)-benzenesulfonyl]-morpholine-3-carboxylic acid hydroxyamide;

(2S,3R,6S)-2,6-dimethyl-4-[4-(pyridin-3-ylmethoxy)-benzenesulfonyl]-morpholine-3-carboxylic acid hydroxyamide;

(2S,3R,6S)-2,6-dimethyl-4-[4-(2-methyl-pyridin-3-ylmethoxy)-benzenesulfonyl]-20 morpholine-3-carboxylic acid hydroxyamide;

(3R,6S)-2,2,6-trimethyl-4-[4-(2-trifluoromethyl-benzyloxy)-benzenesulfonyl]-morpholine-3-carboxylic acid hydroxyamide;

(2S,3R)-2,6,6-trimethyl-4-[4-(pyridin-4-ylmethoxy)-benzenesulfonyl]-morpholine-3-carboxylic acid hydroxyamide;

(3R,6S)-2,2,6-trimethyl-4-[4-(2-methyl-pyridin-3-ylmethoxy)-benzenesulfonyl]-morpholine-3-carboxylic acid hydroxyamide;

(2S,3R,6S)-[4-(2,5-dimethyl-benzyloxy)-benzenesulfonyl]-2,6-dimethyl-morpholine-3-carboxylic acid hydroxyamide;

(2S,3H,6S)-4-[4-(3,5-difluoro-benzyloxy)-benzenesulfonyl]-2,6-dimethyl-morpholine-3-30 carboxylic acid hydroxyamide;

(2S,3R,6S)-4-[4-(3-methoxy-benzyloxy)-benzenesulfonyl]-2,6-dimethyl-morpholine-3-carboxylic acid hydroxyamide;

(2S,3R,6S)-4-[4-(5-fluoro-2-methyl-benzyloxy)-benzenesulfonyl]-2,6-dimethyl-morpholine-3-carboxylic acid hydroxyamide;

(2S,3R,6S)-4-[4-(furan-3-ylmethoxy)-benzenesulfonyl]-2,6-dimethyl-morpholine-3-carboxylic acid hydroxyamide;

(2S,3R,6S)-4-[4-(2-fluoro-3-methyl-benzyloxy)-benzenesulfonyl]-2,6-dimethyl-morpholine-3-carboxylic acid hydroxyamide;

(2S,3R)-4-[4-(4-fluoro-benzyloxy)-benzenesulfonyl]-2,6,6-trimethyl-morpholine-3-carboxylic acid hydroxyamide;

(3R)-4-[4-(4-fluoro-benzyloxy)-benzenesulfonyl]-6,6-dimethyl-morpholine-3-carboxylic acid hydroxyamide;

(3R)-6,6-dimethyl-4-[4-(pyridin-4-ylmethoxy)-benzenesulfonyl]-morpholine-3-carboxylic acid hydroxyamide;

(3R)-6,6-dimethyl-4-[4-(2-methyl-benzyloxy)-benzenesulfonyl]-morpholine-3-carboxylic acid hydroxyamide;

(2S,3R,6S)-4-(4-cyclohexylmethoxy-benzenesulfonyl)-2,6-dimethyl-morpholine-3-carboxylic acid hydroxyamide;

(3R,6S)-4-[4-(2,5-dimethyl-benzyloxy)-benzenesulfonyl]-2,2,6-trimethyl-morpholine-3-carboxylic acid hydroxyamide;

(2S,3R)-4-[4-(4-fluoro-benzyloxy)-benzenesulfonyl]-6-methoxymethyl-2-methyl-morpholine-3-carboxylic acid hydroxyamide;

(2S,3R,6S)-4-[4-(3-chloro-benzyloxy)-benzenesulfonyl]-6-[(ethyl-methyl-amino)-methyl]-2-methyl-morpholine-3-carboxylic acid hydroxyamide;

(2S,3R)-4-[4-(3-chloro-benzyloxy)-benzenesulfonyl]-6-methoxy-2-methyl-morpholine-3-carboxylic acid hydroxyamide;

(2S,3R,6R)-4-[4-(4-fluoro-benzyloxy)-benzenesulfonyl]-6-hydroxymethyl-2-methyl-morpholine-3-carboxylic acid hydroxyamide.

Another embodiment of the invention is that group of composition and method combinations wherein one of the active ingredients of said combination is a arylsulfonyl hydroxamic acid derivative TACE inhibitor wherein said arylsulfonyl hydroxamic acid derivative has the formula of:

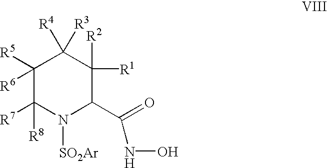

wherein R¹-R⁸ are selected from the group consisting of hydroxy, hydrogen, NH₂, halogen, —CN, (C₁-C₆)alkyl, (C₂-C₆)alkenyl, (C₆-C₁₀)aryl(C₂-C₆)alkenyl, (C₂-C₉)heteroaryl(C₂-C₆)alkenyl, (C₂-C₆)alkynyl, (C₆-C₁₀)aryl(C₂-C₆)alkynyl, (C₂-C₉)heteroaryl(C₂-C₆)alkynyl, (C₁-C₆)alkylamino, [(C₁-C₆)alkyl]₂amino, (C₁-C₆)alkylthio, (C₁-C₆)alkoxy, perfluoro(C₁-C₆)alkyl, perfluoro(C₁-C₆)alkoxy, (C₆-C₁₀)aryl, (C₂-C₉)heteroaryl, (C₆-C₁₀)arylamino, (C₆-C₁₀)arylthio, (C₆-C₁₀)aryloxy, (C₂-C₉)heteroarylamino, (C₂-C₉)heteroarylthio, (C₂-C₉)heteroaryloxy, (C₃-C₆)cycloalkyl, (C₁-C₆)alkyl(hydroxymethylene), piperidyl, (C₁-C₆)alkylpiperidyl, (C₁-C₆)acyl, (C₁-C₆)acylamino, (C₁-C₆)acylthio, (C₁-C₆)acyloxy, (C₁-C₆)alkoxy—(C═O)—, —CO₂H, (C₁-C₆)alkyl—NH—(C═O)—, and [(C₁-C₆)alky]₂—N—(C═O)—;

wherein said (C₁-C₆)alkyl is optionally substituted by one or two groups selected from (C₁-C₆)alkylthio, (C₁-C₆)alkoxy, trifluoromethyl, halo, —CN, (C₆-C₁₀)aryl, (C₂-C₉)heteroaryl, (C₆-C₁₀)arylamino, (C₆-C₁₀)arylthio, (C₆-C₁₀)aryloxy, (C₂-C₉)heteroarylamino, (C₂-C₉)heteroarylthio, (C₂-C₉)heteroaryloxy, (C₆-C₁₀)aryl (C₆-C₁₀)aryl, (C₃-C₆)cycloalkyl, hydroxy, piperazinyl, (C₆-C₁₀)aryl(C₁-C₆)alkoxy, (C₂-C₉)heteroaryl(C₁-C₆)alkoxy, (C₁-C₆)acylamino, (C₁-C₆)acylthio, (C₁-C₆)acyloxy, (C₁-C₆)alkylsulfinyl, (C₆-C₁₀)arylsulfinyl, (C₁-C₆)alkylsulfonyl, (C₆-C₁₀)arylsulfonyl, amino, (C₁-C₆)alkylamino or ((C₁-C₆)alkyl)₂amino;

or R¹ and R², or R³ and R⁴, or R⁵ and R⁶ may be taken together to form a carbonyl;

or R¹ and R², or R³ and R⁴, or R⁵ and R⁶, or R⁷ and R⁸ may be taken together to form a (C₃-C₆)cycloalkyl, oxacyclohexyl, thiocyclohexyl, indanyl or tetralinyl ring or a group of the formula

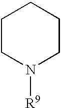

R⁹ is hydrogen or (C₁-C₆)alkyl;

Ar is (C₆-C₁₀)aryl(C₁-C₆)alkoxy(C₆-C₁₀)aryl, (C₆-C₁₀)aryl(C₁-C₆)alkoxy(C₂-C₉)heteroaryl, (C₂-C₉)heteroaryl(C₁-C₆)alkoxy(C₆-C₁₀)aryl, (C₂-C₉)heteroaryl(C₁-C₆)alkoxy(C₂-C₉)heteroaryl optionally substituted by one or more substituents, independently selected from halo, —CN, (C₁-C₆)alkyl optionally substituted with one or more fluorine atoms, hydroxy, hydroxy-(C₁-C₆)alkyl, (C₁-C₆)alkoxy optionally substituted with one or more fluorine atoms, (C₁-C₆)alkoxy(C₁-C₆)alkyl, HO—(C═O)—, (C₁-C₆)alkyl—O—(C═O)—, HO—(C═O)—(C₁-C₆)alkyl, (C₁-C₆)alkyl—O—(C═O)—(C₁-C₆)alkyl, (C₁-C₆)alkyl—(C═O)—O—, alkyl; H(O═C)—, H(O═C)—(C₁-C₆)alkyl, (C₁-C₆alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl—NH—(C═O)—, [(C₁-C₆)alkyl]₂N—(C═O)—, H₂N(C═O)—(C₁-C₆)alkyl, (C₁-C₆)alkyl—HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH—, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl—S—, (C₁-C₆)alkyl-(S═O)—, (C₁-C₆)alkyl—SO₂—, (C₁-C₆)alkyl—SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO—₂—(C₁-C₆)alkyl, CF₃SO₃—, (C₁-C₆)alkyl—SO₃—, phenyl, phenyl(C₁-C₆)alkyl, (C₃-C₁₀)cycloalkyl, (C₂-C₉)heterocycloalkyl, and (C₂-C₉)heteroaryl;

Another embodiment of the invention is that group of composition and method combinations wherein one of the active ingredients of said combination is an arylsulfonyl hydroxamic acid derivative TACE inhibitor wherein said TACE inhibitor is selected from the group consisting of:

(2R,5R)-1-[4-(2,5-Dimethyl-benzyloxy)-benzenesulfonyl]-5-hydroxy-piperidine-2-carboxylic acid hydroxyamide;

(2R,5R)-1-[4-(5-Fluoro-2-methyl-benzyloxy)-benzenesulfonyl]-5-hydroxy-piperidine-2-carboxylic acid hydroxyamide;

(2R,4R)-4-Hydroxy-1-[4-(2-methyl-benzyloxy)-benzenesulfonyl]-piperidine-2-carboxylic acid hydroxyamide;

(2R,5R)-1-[4-(5-Fluoro-2-trifluoromethyl-benzyloxy)-benzenesulfonyl]-5-hydroxy-piperidine-2-carboxylic acid hydroxyamide;

(2R,5R)-5-Hydroxy-1-[4-(2-isopropyl-benzyloxy)-benzenesulfonyl]-piperidine-2-carboxylic acid hydroxyamide;

(2R,5R)-1-[4-(2-Ethyl-benzyloxy)-benzenesulfonyl]-5-hydroxy-piperidine-2-carboxylic acid hydroxyamide;

(2R,4R)-1-[4-(5-Fluoro-2-methyl-benzyloxy)-benzenesulfonyl]-4-hydroxy-piperidine-2-carboxylic acid hydroxyamide;

(2R,4R)-1-[4-(2,5-Dimethyl-benzyloxy)-benzenesulfonyl]-4-hydroxy-piperidine-2-carboxylic acid hydroxyamide;

(2R,5R)-1-[4-(5-Fluoro-2-methyl-benzyloxy)-benzenesulfonyl]-5-hydroxy-5-methyl-piperidine-2-carboxylic acid hydroxyamide;

(2R,5R)-1-[4-(5-Fluoro-2-trifluoromethyl-benzyloxy)-benzenesulfonyl]-5-hydroxy-5-methyl-piperidine-2-carboxylic acid hydroxyamide;

(2R,5R)-5-Hydroxy-1-[4-(2-isopropyl-benzyloxy)-benzenesulfonyl]-5-methyl-piperidine-2-carboxylic acid hydroxyamide;

(2R,5R)-5-Hydroxy-5-methyl-1-[4-(2-methyl-benzyloxy)-benzenesulfonyl]-piperidine-2-carboxylic acid hydroxyamide;

(2R,3R,5R)-5-Hydroxy-3-methyl-1-[4-(2-methyl-benzyloxy)-benzenesulfonyl]-piperidine-2-carboxylic acid hydroxyamide;

(2R,3R,5R)-5-Hydroxy-1-[4-(2-isopropyl-benzyloxy)-benzenesulfonyl]-3-methyl-piperidine-2-carboxylic acid hydroxyamide;

(2R,3S)-1-[4-(5-Fluoro-2-trifluoromethyl-benzyloxy)-benzenesulfonyl]-3-hydroxy-3-methyl-piperidine-2-carboxylic acid hydroxyamide;

(2R,3R)-1-[4-(2,4-dichloro-benzyloxy)-benzenesulfonyl]-3-hydroxy-3-methyl-piperidine-2-carboxylic acid hydroxyamide;

(2R,5R)-1-[4-(2,4-dichloro-benzyloxy)-benzenesulfonyl]-5-hydroxy-3,3-dimethyl-piperidine-2-carboxylic acid hydroxyamide;

(2R,3S)-1-[4-(2-methyl-benzyloxy)-benzenesulfonyl]-4-aminoacetyl-3-methyl-piperazine-2-carboxylic acid hydroxyamide;

(2R,3S)-1-[4-(4-fluoro-2-methyl-benzyloxy)-benzenesulfonyl]-3-methyl-5-oxo-piperazine-2-carboxylic acid hydroxyamide;

(2R,3S)-4-[4-(2-ethyl-benzyloxy)-benzenesulfonyl]-3-methyl-4-carboxylic acid methylamide-piperazine-2-carboxylic acid hydroxyamide;

(2R,3R)-1-[4-(4-fluoro-2-methyl-benzyloxy)-benzenesulfonyl]-3-hydroxy-3-methyl-piperidine-2-carboxylic acid hydroxyamide;

(2R,5R)-1-[4-(2-chloro-4-fluoro-benzyloxy)-benzenesulfonyl-5-hydroxy-3,3-dimethyl-piperidine-2-carboxylic acid hydroxyamide;

(2R,3S)-4-[4-(5-fluoro-2-methyl-benzyloxy)-benzenesulfonyl]-3-methyl-4-carboxylic acid methylamide-piperazine-2-carboxylic acid hydroxyamide;

(2R,3R)-1-[4-(2-chloro-4-fluoro-benzyloxy)-benzenesulfonyl]-3-hydroxy-3-methyl-piperidine-2-carboxylic acid hydroxyamide;

(2R,3R)-1-[4-(2-fluoro-4-chloro-benzyloxy)-benzenesulfonyl]-3-hydroxy-3-methyl-piperidine-2-carboxylic acid hydroxyamide;

(2R,5R)-1-[4-(4-fluoro-benzyloxy)-benzenesulfonyl]-5-hydroxy-3,3-dimethyl-piperidine-2-carboxylic acid hydroxyamide;

(2R,3S)-1-[4-(2-methyl-5-fluoro-benzyloxy)-benzenesulfonyl]-3-methyl-5-oxo-piperazine-2-carboxylic acid hydroxyamide;

(2R,3S)-1-[4-(2-methyl-benzyloxy)-benzenesulfonyl]-3-hydroxy-3-methyl-piperidine-2-carboxylic acid hydroxyamide;

(2R,5R)-1-[4-(4-fluoro-2-methyl-benzyloxy)-benzenesulfonyl]-5-hydroxy-3,3-dimethyl-piperidine-2-carboxylic acid hydroxyamide;

(2R,5R)-1-[4-(2-methyl-3-fluoro-benzyloxy)-benzenesulfonyl]-5-hydroxy-3,3-dimethyl-piperidine-2-carboxylic acid hydroxyamide;

(2R,3R)-1-[4-(2-fluoro-benzyloxy)-benzenesulfonyl]-3-hydroxy-3-methyl-piperidine-2-carboxylic acid hydroxyamide;

(2R,3R)-1-[4-(2-chloro-benzyloxy)-benzenesulfonyl]-3-hydroxy-3-methyl-piperidine-2-carboxylic acid hydroxyamide;

(2R,3R)-1-[4-(2-methyl-3-fluorobenzyloxy)-benzenesulfonyl]-3-hydroxy-3-methyl-piperidine-2-carboxylic acid hydroxyamide;

(2R,5R)-1-[4-(2-methyl-5-chloro-benzyloxy)-benzenesulfonyl]-5-hydroxy-3,3-dimethyl-piperidine-2-carboxylic acid hydroxyamide;

(2R,3R)-1-[4-(2-methyl-benzyloxy)-benzenesulfonyl]-3-hydroxy-3-methyl-piperidine-2-carboxylic acid hydroxyamide;

(2R,3R)-1-[4-(2,4-difluoro-benzyloxy)-benzenesulfonyl]-3-hydroxy-3-methyl-piperidine-2-carboxylic acid hydroxyamide;

(2R,5R)-1-[4-(2-fluoro-5-chloro-benzyloxy)-benzenesulfonyl]-5-hydroxy-3,3-dimethyl-piperidine-2-carboxylic acid hydroxyamide;

(2R,3R)-1-[4-(2-methyl-5-fluorobenzyloxy)-benzenesulfonyl]-3-hydroxy-3-methyl-piperidine-2-carboxylic acid hydroxyamide;

(2R, 5R)-1-[4-(2-bromo-benzyloxy)-benzenesulfonyl]-5-hydroxy-3,3-dimethyl-piperidine-2-carboxylic acid hydroxyamide; and

(2R,3S)-4-[4-(2 ,4-difluoro-benzyloxy)-benzenesulfonyl]-3-methyl-4-carboxylic acid methylamide-piperazine-2-carboxylic acid hydroxyamide.

The methods and compositions of the present invention are generally directed toward treatment and/or prophylaxis of IL-1/18 and TNF mediated diseases in mammals. While any mammal that suffers from IL-1/18 and TNF mediated diseases may be treated using the compositions and methods of the present invention, preferably, the mammal is human.

While the methods and compositions of the present invention are useful for treatment of any IL-1/18 and TNF mediated diseases, preferably, the IL-1/18 and TNF mediated disease may be inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. Preferably, the compositions and methods of treatment are directed to inflammatory disorders such as rheumatoid arthritis, osteoarthritis, septic shock, COPD and periodontal disease.

Combinations of IL-1inhibitors with a TNF inhibitor may also be useful in the treatment of bone and cartilage resorption as well as diseases resulting in excess deposition of extracellular matrix. Such diseases include osteoporosis, periodontal diseases, interstitial pulmonary fibrosis, cirrhosis, systemic sclerosis and keloid formation. Combinations of IL-1 inhibitors with a TNF inhibitor may also be useful in treatment of certain tumors which produce IL-1 as an autocrine growth factor and in preventing the cachexia associated with certain tumors. Combinations of IL-1 inhibitors with a TNF inhibitor may also be useful in the treatment of neuronal diseases with an inflammatory component, including, but not limited to Alzheimer's disease, stroke, depression and percussion injury. Combinations of IL-1 inhibitors with a TNF inhibitor may also be useful in treating cardiovascular diseases in which recruitment of monocytes into the subendothelial space plays a role, such as the development of atherosclerotic plaques.

Diseases for which the methods and compositions are particularly useful are arthritis, and particularly, rheumatoid arthritis.

The present invention also provides a kit comprising in one or more containers a combination of an agent that inhibits the propagation of IL-1 with a TNF inhibitor for treating inflammation.

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

“IL-1 inhibitor” refers to any substance that prevents progation of the IL-1 signal, such as the post-translational processing and release of IL-1 cytokines such as by preventing cleavage of the 31 kDal pro-cytokines that are precursors to the carboxy-terminal 17 kDal mature cytokines, or by preventing release of the mature cytokines into the cellular and/or extracellular fluids. Examples of such inhibitors are inhibitors of ICE, inhibitors of caspase, and inhibitors of IL-1 post-translational processing.

“IL-18 inhibitor” refers to any substance that prevents the propagation of the IL-18 signal such as IL-18 antagonists, IL-18 and IL-18r antibodies and soluble IL-18 receptors (IL-18sr), such as by preventing cleavage of the precursor protein, for example by caspase-1 or caspase-4, thus preventing the liberation of the 156 amino acid mature protein.

“TNF inhibitor” refers to any substance that prevents the propagation of the TNF signal such as TNF antagonists; TNF, TNFr and TACE antibodies; soluble TNF receptors (TNFsr); and TACE inhibitors.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS-STRUCTURE AND MOLECULAR PROPERTIES, 2nd Ed., T. E. Creighton, W. H. Freeman and Company, N.Y., 1993 and Wold, F., Post-translational Protein Modifications: Perspectives and Prospects, pgs. 1-12 in POST-TRANSLATIONAL COVALENT MODIFICATION OF PROTEINS, B. C. Johnson, Ed., Academic Press, N.Y., 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:48-62.

“Variant” as the term is used herein, is a polypeptide that differs from a reference polypeptide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polypeptide may be naturally occurring or it may be a variant that is not known to occur naturally. Non-naturally occurring variants of polynucleotides and polypeptides may be made by mutagenesis techniques or by direct synthesis.

“Identity” is a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY; Lesk, A. M., ed., Oxford University Press, N.Y., 1988; BIOCOMPUTING: INFORMATICS AND GENOME PROJECTS, Smith, D. W., ed., Academic Press, N.Y., 1993; COMPUTER ANALYSIS OF SEQUENCE DATA, PART I, Griffin, A. M., and Griffin, H. G., eds., Humana Press, N.J., 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, N.Y., 1991). While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J Applied. Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12 (1):387), BLASTP, BLASTN, FASTA (Atschul, S. F. et al., J Molec Biol (1990) 215:403).

The term “isolated protein” or “isolated polypeptide” is a protein or polypeptide that by virtue of its origin or source of derivation (1) is not associated with naturally associated components that accompany it in its native state, (2) is free of other proteins from the same species (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

A protein or polypeptide is “substantially pure” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% WAN of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally-occurring sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

The term “polypeptide analog” as used herein refers to a polypeptide that is comprised of a segment of at least 25 amino acids that has substantial identity to a portion of an amino acid sequence and that has at least one of the following properties: (1) specific binding to IL-1, IL-1r, IL-18, IL-18r, TNF, TNFr or TACE under suitable binding conditions, (2) ability to block IL-1, IL-18, TNF or TACE or IL-1, IL-18 or TNF binding to IL-1r, IL-18r or TNFr, or (3) ability to reduce IL-1r, IL-18r or TNFr cell surface expression. Typically, polypeptide analogs comprise a conservative amino acid substitution (or insertion or deletion) with respect to the naturally-occurring sequence. Analogs typically are at least 20 amino acids long, preferably at least 50 amino acids long or longer, and can often be as long as a full-length naturally-occurring polypeptide.

Non-peptide analogs are commonly used in the pharmaceutical industry as drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as κ and λ light chains. Heavy chains are classified as μ, Δ, Υ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin, or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. An Fab fragment is a monovalent fragment consisting of the VL, VH, CL and CH I domains; a F(ab′)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain. A single-chain antibody (scFv) is an antibody in which a VL and VH regions are paired to form a monovalent molecules via a synthetic linker that enables them to be made as a single protein chain (Bird et al., Science 242:423-426, 1988 and Huston et al., Proc. Natl. Acad. Sci. U.S.A. 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. U.S.A. 90:6444-6448, 1993, and Poljak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler & Milstein, Nature 256:495 (1975), or may be made by recombinant DNA methods [see, e.g. U.S. Pat. No. 4,816,567 (Cabilly et al.)].

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity [U.S. Pat. No. 4,816,567; Cabilly et al.; Morrison et al., Proc. Natl. Acad. Sci. U.S.A. 81, 6851-6855 (1984)].

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. Examples of isolated antibodies include an anti-(IL-1, IL-1r, IL-18, IL-18r, TNF, TNFr or TACE) antibody that has been affinity purified using IL-1, IL-1r, IL-18, IL-18r, TNF, TNFr or TACE as an isolated antibody, an anti-(IL-1, IL-1r, IL-18, IL-18r, TNF, TNFr or TACE) antibody that has been synthesized by a hybridoma or other cell line in vitro, and a human anti-(IL-1, IL-1r, IL-18, IL-18r, TNF, TNFr or TACE) antibody derived from a transgenic mouse.

The term “human antibody” includes all antibodies that have one or more variable and constant regions derived from human immunoglobulin sequences. These antibodies may be prepared in a variety of ways, as described below.

A humanized antibody is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In a preferred embodiment, one or more of the CDRs are derived from a human anti-(IL-1, IL-1r, IL-18, IL-18r, TNF, TNFr or TACE) antibody. In a more preferred embodiment, all of the CDRs are derived from a human anti-(IL-1, IL-1r, IL-18, IL-18r, TNF, TNFr or TACE) antibody. In another preferred embodiment, the CDRs from more than one human anti-(IL-1, IL-1r, IL-18, IL-18r, TNF, TNFr or TACE) antibodies are mixed and matched in a chimeric antibody. For instance, a chimeric antibody may comprise a CDR1 from the light chain of a first human anti-(IL-1, IL-1r, IL-18, IL-18r, TNF, TNFr or TACE) antibody may be combined with CDR2 and CDR3 from the light chain of a second human anti-(IL-1, IL-1r, IL-18, IL-18r, TNF, TNFr or TACE) antibody, and the CDRs from the heavy chain may be derived from a third anti-(IL-1, IL-1r, IL-18, IL-18r, TNF, TNFr or TACE) antibody. Further, the framework regions may be derived from one of the same anti-(IL-1, IL-1r, IL-18, IL-18r, TNF, TNFr or TACE) antibodies, from one or more different human antibodies, or from a humanized antibody.

A “neutralizing antibody” or “an inhibitory antibody” is an antibody that inhibits the binding of IL-1, IL-18, TNF or TACE to IL-1r, IL-18r or TNFr when an excess of the anti-(IL-1, IL-1r, IL-18, IL-18r, TNF, TNFr or TACE) antibody reduces the amount of IL-1, IL-18 or TNF bound to IL-1r, IL-18r or TNFr by at least about 20%. In a preferred embodiment, the antibody reduces the amount of binding by at least 40%, more preferably 60%, even more preferably 80%, or even more preferably 85%. The binding reduction may be measured by any means known to one of ordinary skill in the art, for example, as measured in an in vitro competitive binding assay.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “K_{off}” refers to the off rate constant for dissociation of an antibody from the antibody/antigen complex.

The term “K_{d}” refers to the dissociation constant of a particular antibody-antigen interaction.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM, preferably ≦100 nM and most preferably <10 nM.

Fragments or analogs of antibodies or immunoglobulin molecules can be readily prepared by those of ordinary skill in the art following the teachings of this specification. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, ρ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucle6tides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (i99i); Zon et al. Oligonucleotides and Analogues: A Practical Approach, pp. 87-1-08 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

Unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6× SSPE or SSC, 50% formamide, 5× Denhardt's reagent, 0.5% SDS, 100 μpig/ml denatured, fragmented salmon sperm DNA at a hybridization temperature of 42° C. for 12-16 hours, followed by twice washing at 55° C. using a wash buffer of 1× SSC, 0.5% SDS. See also Sambrook et al., supra, pp. 9.50-9.55.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is identical to all or a portion of a reference polynucleotide sequence, or that a polypeptide sequence is identical to a reference polypeptide sequence. In contrast, the term “complementary to” is used herein to mean that the complementary sequence is identical to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 98 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, even more preferably at least 98 percent sequence identity and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is iysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

The term “agent” is used herein to denote a chemical compound, a mixture of chemical compounds, a biological macromolecule, or an extract made from biological materials.

The term patient includes human and veterinary subjects.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

“Administering” means administering a first agent and while that agent is becoming active or still active, administering a second agent; either of the two agents may be the first to be administered, and the two agents may be administered simultaneously. For example, administering an IL-1 processing and release inhibiting agent and TACE inhibitor to a mammal may be accomplished by first administering the IL-1 processing and release inhibiting agent, and then before or within the time that the IL-1 processing and release inhibiting agent reaches its maximum concentration in the body fluids of the mammal, administering TACE inhibitor, or by first administering the IL-1 processing and release inhibiting agent and then administering the TACE inhibitor, or by administering the IL-1 processing and release inhibiting agent together with the TACE inhibitor.

The term “alkyl”, as used herein, unless otherwise indicated, includes saturated monovalent hydrocarbon radicals having straight, branched or cyclic moieties or combinations thereof.

The term “alkoxy”, as used herein, includes O-alkyl groups wherein “alkyl” is defined above.

The term “cycloalkyl”, as used herein, includes (C₃-C₁₄) mono-, bi- and tri-cyclic saturated hydrocarbon compounds, optionally substituted by 1 to 2 substituents selected from the group consisting of hydroxy, fluoro, chloro, trifluoromethyl, (C₁-C₆)alkoxy, (C₆-C₁₀)aryloxy, trifluoromethoxy, difluoromethoxy and (C₁-C₆)alkyl. Preferably, cycloalkyl is substituted with hydroxy.

The term “aryl”, as used herein, unless otherwise indicated, includes an organic radical derived from an aromatic hydrocarbon by removal of one hydrogen, such as phenyl or naphthyl, optionally substituted by 1 to 3 substituents selected from the group consisting of fluoro, chloro, trifluoromethyl, (C₁-C₆)alkoxy, (C₆-C₁₀)aryloxy, trifluoromethoxy, difluoromethoxy and (C₁-C₆)alkyl.

The term “heteroaryl”, especially (C₅-C₉), as used herein, unless otherwise indicated, includes an organic radical derived from an aromatic heterocyclic compound (e.g., 5 to 9 membered mono or bicyclic ring containing one or more heteroatoms) by removal of one hydrogen, such as pyridyl, furyl, pyroyl, thienyl, isothiazolyl, imidazolyl, benzimidazolyl, tetrazolyl, pyrazinyl, pyrimidyl, quinolyl, isoquinolyl, benzofuryl, isobenzofuryl, benzothienyl, pyrazolyl, indolyl, isoindolyl, purinyl, carbazolyl, isoxazolyl, thiazolyl, oxazolyl, benzthiazolyl or benzoxazolyl, optionally substituted by 1 to 2 substituents selected from the group consisting of fluoro, chloro, trifluoromethyl, (C₁-C₆)alkoxy, (C₆-C₁₀)aryloxy, trifluoromethoxy, difluoromethoxy and (C₁-C₆)alkyl.

The term “acyl”, as used herein, unless otherwise indicated, includes a radical of the general formula RCO wherein R is alkyl, alkoxy, aryl, arylalkyl or arylalkyloxy and the terms “alkyl” or “aryl” are as defined above.

The term “acyloxy”, as used herein, includes O-acyl groups wherein “acyl” is defined above.

“Incorporation by reference” as used herein means incorporation not only of the text and graphics of the reference, but also the preferences, genera, subgenera, and specific embodiments of the reference.

### DETAILED DESCRIPTION

The present invention is directed to compositions comprising a combination of an agent that inhibits the propagation of Interleukin-1 (IL-1) and/or IL-18 with a Tumor Necrosis Factor (TNF) inhibitor for treating inflammation, including rheumatoid arthritis.

Inhibitors of the propagation of the IL-1/18 response include soluble IL-1/18 receptors, antibodies to IL-1, IL-1r, IL-18 and IL-18r; IL-1ra polypeptides and IL-1 processing and release inhibiting agents, preferably IL-1 processing and release inhibiting agents. TNF inhibitors include soluble TNF receptors, TNF antibodies (to TNF or its receptor) and TACE inhibitors, particularly TACE inhibitors. These combinations provide an unexpected synergy due to the fact that the biological effects of these cytokines, although overlapping, are not identical.

IL-1ra

IL-1ra polypeptides and analogs are well known in the art, and those skilled in the art understand how to make and use them for treatment of disease. The polypeptides useful in the present invention include but are not limited to those described in the following references. The most preferred IL-1ra is anakinra (Kinerete) U.S. Pat. Nos. 5,872,095, 5,874,561 and 5,824,549 describe methods of treating diseases using IL-1 receptor antagonist proteins and methods for generating IL-1 receptor antagonist proteins. U.S. Pat. Nos. 5,872,095, 5,874,561 and 5,824,549 are hereby incorporated by reference in their entirety for all purposes as if fully set forth herein.

U.S. Pat. No. 5,874,561 describes various IL-1 receptor antagonist proteins, as well as methods for making them and therapeutic methods using them. U.S. Pat. No. 5,874,561 is hereby incorporated by reference in its entirety for all purposes as if fully set forth herein.

U.S. Pat. No. 5,455,330 describes a particular class of IL-1 receptor antagonist proteins, as well as methods for making them and therapeutic methods using them. U.S. Pat. No. 5,455,330 is hereby incorporated by reference in its entirety for all purposes as if fully set forth herein.

U.S. Pat. No. 5,075,022 describes the structure, properties and methods of making IL-1ra, and in particular, its corresponding DNA sequence. U.S. Pat. No. 5,075,022 is hereby incorporated by reference in its entirety for all purposes as if fully set forth herein.

Preferred polypeptides that are useful in the present invention include the polypeptide of SEQ ID NO: 2 of U.S. Pat. No. 5,863,769 which is incorporated herein by reference in its entirety for all purposes as if fully set forth herein. Particularly preferred is the mature IL-1ra beta polypeptide described therein, which differs from the ordinary human IL-1ra in that it incorporates an N-terminal methionin. Moreover, polypeptides are useful which have at least 80% identity to the polypeptide of SEQ ID NO: 2 of U.S. Pat. No. 5,863,769 or the relevant portion and more preferably at least 85% identity, and still more preferably at least 90% identity, and even still more preferably at least 95% identity to SEQ ID NO: 2 of U.S. Pat. No. 5,863,769.

Useful IL-1ra beta polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Thus, the polypeptides particularly useful in the present invention include polypeptides having an amino acid sequence at least identical to that of SEQ ID NO: 2 of U.S. Pat. No. 5,863,769 or fragments thereof with at least 80% identity to the corresponding fragment of SEQ ID NO: 2 of U.S. Pat. No. 5,863,769. Preferably, all of these polypeptides retain the biological activity of the IL-1ra beta, including antigenic activity. Included in this group are variants of the defined sequence and fragments. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gin; and among the basic residues Lys and Arg; or aromatic residues Phe and Tyr. Particularly preferred are variants in which several, 5-10, 1-5, or 1-2 amino acids are substituted, deleted, or added in any combination.

The IL-1ra beta polypeptides that are particularly useful in the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Other preferred polypeptides useful in the present invention also include IL-1ra polypeptides as described above and additionally conjugated with one or more polymeric moieties that protect the IL-1ra polypeptide from enzymatic degradation that may take place in the gut of an animal, in the blood serum or other extracellular environment of an animal, or within the cells of an animal. Preferred polymeric moieties useful for conjugating IL-1ra for the present invention are so-called linear and branched pegylation reagents such as those described in U.S. Pat. Nos. 5,681,811 and 5,932,462, both of which are incorporated herein by reference in their entireties for all purposes as if fully set forth herein. Pegylated IL-1ra polypeptide is described, as well, in PCT publication WO 97/28828. Methods for conjugating polymeric moieties to proteins are well known in the art, and are described, for example, in the patents set forth above in this paragraph, as well as in Poly(Ethylene Glycol) Chemistry: Biotechnical and Biomedical Applications J. M. Harris, Ed., Plenum, N.Y., 1992.

IL-1sr

Soluble IL-1 receptors (IL-1sr), methods for their preparation and pharmaceutical compositions containing them are described in U.S. Pat. Nos. 5,081,228; 5,180,812; 5,767,064; and reissue RE 35,450; and European Patent Publication EP 460,846.

IL-18

IL-18 including its receptor and antibodies and soluble receptor (IL-18sr) thereto are described in International Publications WO/99/37772, WO 00/56771 and WO 01/58956 and European Patent Publications EP 864,585 and EP 974,600.

Interleukin Antibodies

Monoclonal antibodies against IL-1, IL-1r, IL-18 or IL-18r can also be prepared according to XenoMouse™ technology.

The XenoMouse™ is an engineered mouse strain that comprises large fragments of the human immunoglobulin loci and is deficient in mouse antibody production. See, e.g., Green et al. Nature Genetics 7:13-21 (1994) and U.S. patent application Ser. No. 07/466,008, filed Jan. 12, 1990, U.S. patent application Ser. No. 07/610,515, filed Nov. 8, 1990, U.S. patent application Ser. No. 07/919,297, filed Jul. 24, 1992, U.S. patent application Ser. No. 07/922,649, filed Jul. 30, 1992, filed U.S. patent application Ser. No. 08/031,801, filed Mar. 15,1993, U.S. patent application Ser. No. 08/112,848, filed Aug. 27, 1993, U.S. patent application Ser. No. 08/234,145, filed Apr. 28, 1994, U.S. patent application Ser. No. 08/376,279, filed Jan. 20, 1995, U.S. patent application Ser. No. 08/430, 938, Apr. 27, 1995, U.S. patent application Ser. No. 08/464,584, filed Jun. 5, 1995, U.S. patent application Ser. No. 08/464,582, filed Jun. 5, 1995, U.S. patent application Ser. No. 08/463,191, filed Jun. 5, 1995, U.S. patent application Ser. No. 08/462,837, filed Jun. 5, 1995, U.S. patent application Ser. No. 08/486,853, filed Jun. 5, 1995, U.S. patent application Ser. No. 08/486,857, filed Jun. 5, 1995, U.S. patent application Ser. No. 08/486,859, filed Jun. 5, 1995, U.S. patent application Ser. No. 08/462,513, filed Jun. 5, 1995 and U.S. patent application Ser. No. 08/724,752, filed Oct. 2, 1996; and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000.

The XenoMouse™ strains were engineered with yeast artificial chromosomes (YACs) containing 245 kb and 190 kb-sized germline configuration fragments of the human heavy chain locus and kappa light chain locus, respectively, which contained core variable and constant region sequences. Id. The XenoMouse™ produces an adult-like human repertoire of fully human antibodies, and generates antigen-specific human Mabs. A second generation Xenomouse™ contains approximately 80% of the human antibody repertoire through introduction of megabase sized, germline configuration YAC fragments of the human heavy chain loci and kappa light chain loci. See Mendez et al. Nature Genetics 15:146-156 (1997), Green and Jakobovits J. Exp. Med. 188:483-495 (1998), and U.S. patent application Ser. No. 08/759,620, filed Dec. 3, 1996, the disclosures of which are hereby incorporated by reference.

In another embodiment, the non-human animal comprising human immunoglobulin gene loci are animals that have a “minilocus” of human immunoglobulins. In the minilocus approach, an exogenous 1 g locus is mimicked through the inclusion of individual genes from the 1 g locus. Thus, one or more V_{H} genes, one or more D_{H} genes, one or more J_{H} genes, a mu constant region, and a second constant region (preferably a gamma constant region) are formed into a construct for insertion into an animal. This approach is described, inter alia, in U.S. Pat. Nos. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

An advantage of the minilocus approach is the rapidity with which constructs including portions of the 1 g locus can be generated and introduced into animals. However, a potential disadvantage of the minilocus approach is that there may not be sufficient immunoglobulin diversity to support full B-cell development, such that there may be lower antibody production.

In another embodiment, the invention provides a combination comprising IL-1, IL-1r, IL-18 or IL-18r antibodies from non-human, non-mouse animals by immunizing non-human transgenic animals that comprise human immunoglobulin loci. One may produce such animals using the methods described in U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The methods disclosed in these patents may modified as described in U.S. Patent No. 5,994,619. In a preferred embodiment, the non-human animals may be rats, sheep, pigs, goats, cattle or horses.

Non-Hybridoma Host Cells and Methods of Recombinantly Producing Protein

Nucleic acid molecules encoding IL-1, IL-1r, IL-18 or IL-18r antibodies and vectors comprising these antibodies can be used for transformation of a suitable mammalian host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (which patents are hereby incorporated herein by reference).

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

Transgenic Animals

Antibodies of the combination invention can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957. In one embodiment, non-human transgenic animals that comprise human immunoglobulin loci are immunized with IL-1, IL-1r, IL-18 or IL-18r or a portion thereof. One may produce such transgenic animals using methods described in U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. In another embodiment, the transgenic animals may comprise a “minilocus” of human immunoglobulin genes. The methods disclosed above may modified as described in, inter alia, U.S. Patent No. 5,994,619. In a preferred embodiment, the non-human animals may be rats, sheep, pigs, goats, cattle or horses. In another embodiment, the transgenic animals comprise nucleic acid molecules encoding anti-(IL-1, IL-1r, IL-18 or IL-18r) antibodies. In a preferred embodiment, the transgenic animals comprise nucleic acid molecules encoding heavy and light chains specific for IL-1, IL-1r, IL-18 or IL-18r. In another embodiment, the transgenic animals comprise nucleic acid molecules encoding a modified antibody such as a single-chain antibody, a chimeric antibody or a humanized antibody. The anti-(IL-1, IL-1r, IL-18 or IL-18r) antibodies may be made in any transgenic animal. In a preferred embodiment, the non-human animals are mice, rats, sheep, pigs, goats, cattle or horses.

Phage Display Libraries

Recombinant anti-(IL-1, IL-1r, IL-18 or IL-18r) human antibodies of the invention in addition to the anti-(IL-1, IL-1r, IL-18 or IL-18r) antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurfZAP™ phage display kit, catalog no. 240612). There are also other methods and reagents that can be used in generating and screening antibody display libraries (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. U.S.A. 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. U.S.A. 88:7978-7982.

In a preferred embodiment, to isolate human anti-(IL-1, IL-1r, IL-18 or IL-18r) antibodies with the desired characteristics, a human anti-(IL-1, IL-1r, IL-18 or IL-18r) antibody as described herein is first used to select human heavy and light chain sequences having similar binding activity toward IL-1, IL-1r, IL-18 or IL-18r, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using human IL-1, IL-1r, IL-18 or IL-18r as the antigen.

Once initial human VL and VH segments are selected, “mix and match” experiments, in which different pairs of the initially selected VL and VH segments are screened for IL-1, IL-1r, IL-18 or IL-18r binding, are performed to select preferred VL/VH pair combinations. Additionally, to further improve the quality of the antibody, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to IL-1, IL-1r, IL-18 or IL-18r.

Following screening and isolation of an anti-(IL-1, IL-1r, IL-18 or IL-18r) antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

Class Switching

Another aspect of the instant invention is to provide a mechanism by which the class of an anti-(IL-1, IL-1r, IL-18 or IL-18r) antibody may be switched with another. In one aspect of the invention, a nucleic acid molecule encoding VL or VH is isolated using methods well-known in the art such that it does not include any nucleic acid sequences encoding CL or CH. The nucleic acid molecule encoding VL or VH are then operatively linked to a nucleic acid sequence encoding a CL or CH from a different class of immunoglobulin molecule. This may be achieved using a vector or nucleic acid molecule that comprises a CL or CH chain, as described above. For example, an anti-(IL-1, IL-1r, IL-18 or IL-18r) antibody that was originally IgM may be class switched to an IgG. Further, the class switching may be used to convert one IgG subclass to another, e.g., from IgG1 to IgG2.

Antibody Derivatives

One may use the nucleic acid molecules described above to generate antibody derivatives using techniques and methods known to one of ordinary skill in the art.

Humanized Antibodies

As was discussed above in connection with human antibody generation, there are advantages to producing antibodies with reduced immunogenicity. This can be accomplished to some extent using techniques of humanization and display techniques using appropriate libraries. It will be appreciated that murine antibodies or antibodies from other species can be humanized or primatized using techniques well known in the art. See e.g., Winter and Harris Immunol Today 14:43-46 (1993) and Wright et al. Crit. Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085).

Mutated Antibodies

In another embodiment, the nucleic acid molecules, vectors and host cells may be used to make mutated anti-(IL-1, IL-1r, IL-18 or IL-18r) antibodies. The antibodies may be mutated in the variable domains of the heavy and/or light chains to alter a binding property of the antibody. For example, a mutation may be made in one or more of the CDR regions to increase or decrease the K_{d} of the antibody for IL-1, IL-1r, IL-18 or IL-18r, to increase or decrease K_{off}, or to alter the binding specificity of the antibody. Techniques in site-directed mutagenesis are well-known in the art. See, e.g., Sambrook et al. and Ausubel et al., supra. In a preferred embodiment, mutations are made at an amino acid residue that is known to be changed compared to germline in a variable region of an anti-(IL-1, IL-1r, IL-18 or IL-18r) antibody. A mutation may be made in a framework region or constant domain to increase the half-life of the anti-(IL-1, IL-1r, IL-18 or IL-18r) antibody. See, e.g., U.S. patent application Ser. No. 09/375,924, filed Aug. 17, 1999, herein incorporated by reference. A mutation in a framework region or constant domain may also be made to alter the immunogenicity of the antibody, to provide a site for covalent or non-covalent binding to another molecule, or to alter such properties as complement fixation. Mutations may be made in each of the framework regions, the constant domain and the variable regions in a single mutated antibody. Alternatively, mutations may be made in only one of the framework regions, the variable regions or the constant domain in a single mutated antibody.

In one embodiment, there are no greater than ten amino acid changes in either the VH or VL regions of the mutated anti-(IL-1, IL-1r, IL-18 or IL-18r) antibody compared to the anti-(IL-1, IL-1r, IL-18 or IL-18r) antibody prior to mutation. In a more preferred embodiment, there is no more than five amino acid changes in either the VH or VL regions of the mutated anti-(IL-1, IL-1r, IL-18 or IL-18r) antibody, more preferably no more than three amino acid changes. In another embodiment, there are no more than fifteen amino acid changes in the constant domains, more preferably, no more than ten amino acid changes, even more preferably, no more than five amino acid changes.

IL-1 Processing and Release Inhibitors

ICE Inhibitors

U.S. Pat. Nos. 5,656,627, 5,847,135, 5,756,466, 5,716,929 and 5,874,424 disclose several classes of ICE inhibitor compounds characterized by hydrogen-bonding, hydrophobic, and electronegative moieties configured so as to bind to the ICE receptor site. These patents disclose generic combinations of the particular ICE inhibitors with inhibitors and antagonists of cytokines, but does not disclose or suggest the combination of an ICE inhibitor and a TNF inhibitor that provides the unexpected synergy of the compositions and methods of the present invention.

One embodiment of the present invention provides for compositions and methods of treatment using compositions comprising a TNF inhibitor and one or more ICE inhibitor compounds of U.S. Pat. Nos. 5,656,627, 5,847,135, 5,756,466, 5,716,929 and 5,874,424. U.S. Pat. Nos. 5,656,627, 5,847,135, 5,756,466, 5,716,929 and 5,874,424 are incorporated herein by reference in their entireties for all purposes as if fully set forth herein.

U.S. Pat. No. 5,585,357 discloses a class of substituted pyrazole ICE inhibitors. One embodiment of the present invention provides for compositions and methods of treatment using compositions comprising a TNF inhibitor and one or more ICE inhibitor compounds of U.S. Pat. No. 5,585,357. U.S. Pat. No. 5,585,357 is incorporated herein by reference in its entirety for all purposes as if fully set forth.

U.S. Pat. No. 5,434,248 discloses a class of peptidyl aldehyde ICE inhibitors. One embodiment of the present invention provides for compositions and methods of treatment using compositions comprising a TNF inhibitor and one or more ICE inhibitor compounds of U.S. Pat. No. 5,434,248. U.S. Pat. No. 5,434,248 is incorporated herein by reference in its entirety for all purposes as if fully set forth.

U.S. Pat. Nos. 5,462,939 and 5,585,486 disclose a class of peptidic ketone ICE inhibitors. One embodiment of the present invention provides for compositions and methods of treatment using compositions comprising a TNF inhibitor and one or more ICE inhibitor compounds of U.S. Pat. Nos. 5,462,939 and 5,585,486. U.S. Pat. Nos. 5,462,939 and 5,585,486 are incorporated herein by reference in their entireties for all purposes as if fully set forth.

U.S. Pat. No. 5,411,985 discloses gamma-pyrone-3-acetic acid as an ICE inhibitor. One embodiment of the present invention provides for compositions and methods of treatment using compositions comprising a TNF inhibitor and gamma-pyrone-3-acetic acid. U.S. Pat. No. 5,411,985 is incorporated herein by reference in its entirety for all purposes as if fully set forth.

U.S. Pat. No. 5,834,514 discloses a class of halomethyl amides as ICE inhibitors. One embodiment of the present invention provides for compositions and methods of treatment using compositions comprising a TNF inhibitor and one or more ICE inhibitor compounds of U.S. Pat. No. 5,834,514. U.S. Pat. No. 5,834,514 is incorporated herein by reference in its entirety for all purposes as if fully set forth.

U.S. Pat. No. 5,739,279 discloses a class of peptidyl derivatives of 4-amino-2,2-difluoro-8-oxo-1,6-hexanedioic acid as ICE inhibitors. One embodiment of the present invention provides for compositions and methods of treatment using compositions comprising a TNF inhibitor and one or more ICE inhibitor compounds of U.S. Pat. No. 5,739,279. U.S. Pat. No. 5,739,279 is incorporated herein by reference in its entirety for all purposes as if fully set forth.

U.S. Pat. No. 5,843,904 discloses a class of peptidyl ICE inhibitors. One embodiment of the present invention provides for compositions and methods of treatment using compositions comprising a TNF inhibitor and one or more ICE inhibitor compounds of U.S. Pat. No. 5,843,904. U.S. Pat. No. 5,843,904 is incorporated herein by reference in its entirety for all purposes as if fully set forth.

U.S. Pat. No. 5,670,494 discloses a class of substituted pyrimidine ICE inhibitors. One embodiment of the present invention provides for compositions and methods of treatment using compositions comprising a TNF inhibitor and one or more ICE inhibitor compounds of U.S. Pat. No. 5,670,494. U.S. Pat. No. 5,670,494 is incorporated herein by reference in its entirety for all purposes as if fully set forth.

U.S. Pat. No. 5,744,451 discloses a class of substituted glutamic acid ICE inhibitors. One embodiment of the present invention provides for compositions and methods of treatment using compositions comprising a TNF inhibitor and one or more ICE inhibitor compounds of U.S. Pat. No. 5,744,451. U.S. Pat. No. 5,744,451 is incorporated herein by reference in its entirety for all purposes as if fully set forth.

U.S. Pat. No. 5,843,905 discloses a class of substituted glutamic acid ICE inhibitors. One embodiment of the present invention provides for compositions and methods of treatment using compositions comprising a TNF inhibitor and one or more ICE inhibitor compounds of U.S. Pat. No. 5,843,905. U.S. Pat. No. 5,843,905 is incorporated herein by reference in its entirety for all purposes as if fully set forth.

U.S. Pat. No. 5,565,430 discloses a class of azaaspartic acid analogs as ICE inhibitors. One embodiment of the present invention provides for compositions and methods of treatment using compositions comprising a TNF inhibitor and one or more ICE inhibitor compounds of U.S. Pat. No. 5,565,430. U.S. Pat. No. 5,565,430 is incorporated herein by reference in its entirety for all purposes as if fully set forth.

U.S. Pat. Nos. 5,552,400 and 5,639,745 disclose a class of fused-bicyclic lactam ICE inhibitors. One embodiment of the present invention provides for compositions and methods of treatment using compositions comprising a TNF inhibitor and one or more ICE inhibitor compounds of U.S. Pat. Nos. 5,552,400 and 5,639,745. U.S. Pat. Nos. 5,552,400 and 5,639,745 are incorporated herein by reference in their entireties for all purposes as if fully set forth.

IL-1 Stimulus Coupled Posttranslational Processing and Release Inhibitors

The IL-1 stimulus coupled posttranslational processing and release inhibiting agents that are useful in the combinations of the present invention are described above. Particularly useful among the IL-1 processing and release inhibiting agents for the present methods and compositions are diarylsulfonyl urea (DASU) compounds. Such compounds can be prepared according to the methods described in PCT Publication WO 98/32733, published Jul. 30, 1998. U.S. Pat. No. 6,022,984, issued Feb. 8, 2000 refers to other methods for preparation of DASU compounds. International Patent Publication WO 01/19390 published Mar. 22, 2001 refers to combinations of IL-1RA with DASU inhibitors. U.S. Provisional Applications 60/328,254 and 60/301,712, filed Oct. 10, 2001 and Jun. 28, 2001, respectively, refer the treatment of atherosclerosis with DASU inhibitors. Related to these DASU compounds are DASU binding proteins (DBPs) that mediate the cytokine inhibitory activity of these agents. DBPs may be used to screen for structurally unique drugs that disrupt stimulus-coupled post-translational processing. Compounds that bind to the DBPs also may be used as therapeutics in the treatment of inflammatory disorders. DBPs are described in U.S. Provisional Patent Application No. 60/098,448, filed Aug. 31, 1998. One skilled in the art will appreciate that antibodies for the DASU binding proteins can be prepared and would have similar activity to the DASU inhibitors described above. Each of the foregoing patents, publications and applications is hereby incorporated by reference in its entirety for all purposes as if fully set forth.

TNF Inhibitors

TNF inhibitors include the soluble TNF receptor (TNFsr), antibodies to TNF and inhibitors of TACE. Commercial TNF inhibitors useful in the present invention include etanercept (Enbrel®), infliximab (Remicade®), CDP-870 and adalimumab (D2E7). Infliximab and methods describing its production and use are described in U.S. Pat. Nos. 5,698,195 and 5,656,272. Adalimumab and methods describing its production and use are described in International Patent Publication WO 97/29131. Methods of producing humanized antibodies such as CDP-870 are described in European Patent Publications 120694, 460167 and 5165,785.

TNFsr (the soluble TNF receptor, e.g., etanercept) is a cytokine cascade blocker. In vivo, it is produced in response to the same enciting events which cause the elicitation of the agonist TNF such as trauma, sepsis and pancreatitis. It is a single molecule. The recombinant molecule (rTNFsr) can be produced as a dimer thereby increasing receptor-ligand affinity approximately 100 fold. The co-efficient of dissociation for the naturally occurring molecule is 10⁻⁷ while the coefficient of dissociation for the recombinant dimer is 10⁻¹¹ (Oppenheim et al., 1993) thereby requiring a smaller dose as a therapeutic than the naturally occurring molecule. Further, the dimer structure leads to an increase of the half-life to 27 hours in vivo permitting single daily dosing (Mohler, 1994). However, any other means that decreases the coefficient of dissociation for the molecule can be used in the practice of the present invention.

Etanercept and methods describing its production and use are described in U.S. Pat. Nos. 5,395,760, 5,712,155, 5,945,397, 5,344,915, and reissue RE 36,755.

Other TNF inhibitors, including methods of their preparation, are described in European Patent Publication 422,339 and U.S. Pat. No. 6,143,866 which also describe PEGylated and glycosylated variants.

TACE Inhibitors

TNF-α Converting Enzyme (TACE) inhibitors and methods for their preparation and uses thereof are described in International Patent Publications WO 00/09485 and WO 00/09492 both published Feb. 24, 2000, and European Patent Publication EP 1,081,137 published Mar. 7, 2001.

Other TACE inhibitors are described in U.S. Pat. No. 5,830,742.

TNF Antibodies

Other antibodies for TNF, TNFr, TNFbp or TACE can be prepared by methods analogous to those described above for the preparation of IL-1, IL-1r, IL-18 or IL-18r antibodies.

Each of the foregoing patents, publications and applications is hereby incorporated by reference in its entirety.

Blockade of the action of either IL-1/18 or TNF alone is known to be sufficient to significantly inhibit the rheumatoid arthritis inflammatory response in rats and septic shock in baboons. In rodent arthritis, joint swelling has been demonstrated to be maximally inhibited by the administration alone of either IL-1ra or TNFbp in rats that were undergoing a reactivated arthritis induced by peptidoglycan-polysaccharide (PG/PS). In septic shock, baboons that were challenged with Escherichia coli were protected to a similar degree against lethality and hemodynamic alterations by the administration alone of either IL-1 ra or TNFbp.

Unexpectedly, however, treatment of rats undergoing an LPS-reactivated arthritis with a combination of an IL-1/18 inhibitor and TNF inhibitor according to the present invention caused synergistic inhibitory effects on joint swelling. The examples below describe methods for demonstrating the synergistic effect of the inventive combinations (i.e. combination therapy with an IL-1/18 inhibitor and a TNF inhibitor) on treating IL-1/18 and TNF-mediated inflammatory diseases, such as rheumatoid arthritis, adult respiratory distress syndrome (ARDS) and sepsis.

In Vivo Synergystic Effect of Combination

An animal model of rheumatoid arthritis induced by two microbial components (lipopolysaccharide (LPS) and peptidoglycanpolysaccharide (PG/PS) can be used to determine the effect of combination therapy for treatment of arthritis. According to R. L. Wilder in Immunggathoeenetic Mechanisms of Arthritis, Chapter 9 entitled “Experimental Animal Models of Chronic Arthritis,” regarding streptococcal cell wall-induced arthritis, “the clinical, histological and radiological features of the experimental joint disease closely resemble those observed in adult and juvenile arthritis.”

According to the following exemplary experiments, the animal model described in Schwab, Experimental Medicine, 1688-1702, (1987), can be used to induce arthritis in the tarsal joints of normal rats. Briefly, arthritis is induced by the sequential administration of two microbial components: (1) first streptococcal cell wall (SCW) products containing peptidoglycanpolysaccharide (PG/PS) is injected intraarticularly, and (2) twenty-one days later, lipopolysaccharide (LPS) from Salmonella typhimurium, is injected intravenously.

To assess the extent of inflammation during the 72-hour period following the intravenous injection of LPS, the dimensions of the ankle joint is measured at 0, 24, 36, 48, and 72 hours after the reactivation of the arthritis.

The effects of IL-1/18 inhibitor and TNF inhibitor when administered singly and in combination are tested on the development of joint swelling during the reactivation of the arthritis. The inhibitors and vehicle are administered subcutaneously at the nape of the neck at time 0, 2, 6, 12, 18, 24, 30, 36, and 42 hours relative to the intravenous injection of LPS. See also Williams, R. O., Marinova-Mutafchieva, L., Feldmann, M., and Maini, R. N., 2000, “Evaluation of TNF-a and IL-1 blockade in collagen-induced arthritis and comparison with combined anti-TNF-a/anti-CD3 therapy”, J. Immunology, 165:7240-7245; Feige, U., Hu, Y.-L., Gasser, J., Campagnuolo, G., Munyakazi, L., and Bolon, B., 1999, “Anti-interleukin-1 and anti-tumor necrosis factor-a synergistically inhibit adjuvant arthritis in Lewis rats”, Cell. Mol. Life Sci., 57:1457-1470; and Joosten, L. A. B., Helsen, M. M. A., Saxne, T., van de Loo, F. A. J., Heinegard, D., and van den Berg, W. B., 1999, “IL-1ab blockade prevents cartilage and bone destruction in murine type 11 collagen-induced arthritis, whereas TNF-a blockade only ameliorates joint inflammation”, J. Immunology, 163:5049-5055.

Inhibition of ATP Induced Release of IL-1α, IL-1β or IL-18

Mononuclear cells are purified from 100 ml of blood isolated using LSM (Organon Teknika). The heparinized blood (1.5 ml of 1000 units/ml heparin for injection from Apotheconis added to each 50 ml syringe) is diluted with 20 ml of Medium (RMI 1640, 5% FBS, 1% pen/strep, 25 mM HEPES, pH 7.3). 30 ml of the diluted blood is layered over 15 ml of LSM (Organon Teknika) in a 50 ml conical polypropylene centrifuge tube. The tubes are centrifuged at 1200 rpm for 30 minutes in benchtop Sorvall centrifuge at room temperature. The mononuclear cells, located at the interface of the plasma and LSM, are removed, diluted with Medium to achieve a final volume of 50 ml, and collected by centrifugation as above The supernatant is discarded and the cell pellet is washed 2 times with 50 ml of medium. A 10 μl sample of the suspended cells is taken before the second wash for counting; based on this count the washed cells are diluted with medium to a final concentration of 2.0×106 cells/ml.

0.1 ml of the cell suspension is added to each well of 96 well plates. The monocytes are allowed to adhere for 2 hours, then non-adherent cells are removed by aspiration and the attached cells are washed twice with 100 μl f Medium. 100 μl of Medium is added to each well, and the cells are incubated overnight at 37EC in a 5% carbon dioxide incubator.

The following day, 25 μl of 50 ng/ml LPS (in Medium) is added to each well and the cells are activated for 2 hours at 37C.

Test agent solutions are prepared as follows. IL-1 processing and release inhibitors are diluted with dimethyl sulfoxide to a final concentration of 10 mM. From this stock solution IL-1 processing and release inhibitors are first diluted 1:50 [5 μl of 10 mM stock+245 μl Chase Medium (RPMI 1640, 25 mM Hepes, pH 6.9, 1% FBS, 1% pen/strep, 10 ng/ml LPS and 5 mM sodium bicarbonate] to a concentration of 200 μM. A second dilution is prepared by adding 10 μl of the 200 μM IL-1 processing and release inhibitor solution to 90 μl of Chase Medium.

The LPS-activated monocytes are washed once with 100 μl of Chase Medium then 100 μl of Chase Medium (containing 0.2% dimethyl sulfoxide) is added to each well. 0.011 ml of the test agent solutions are added to the appropriate wells, and the monocytes are incubated for 30 minutes at 37° C. At this point 2 mM ATP is introduced by adding 12 μl of a 20 mM stock solution (previously adjusted to pH 7.2 with sodium hydroxide) and the cells are incubated for an additional 3 hours at 37° C.

The 96-well plates are centrifuged for 10 minutes at 2000 rpm in a Sorvall benchtop centrifuge to remove cells and cell debris. A 90 μl aliquot of each supernatant is removed and transferred to a 96 well round bottom plate and this plate is centrifuged a second time to ensure that all cell debris is removed. 30 μl of the resulting supernatant is added to a well of an IL-1 β ELISA plate that also contains 70 μl of PBS, 1% FBS. The ELISA plate is incubated overnight at 4° C. The ELISA (R&D Systems) is run following the kit directions.

Data Calculation and Analysis:

The amount of IL-1 β immunoreactivity in the Chase medium samples is calculated as the percent control, which equals one hundred times the quotient of the difference between optical density at 450 nm of the test compound well and the optical density at 450 nm of the Reagent Blank wells on the ELISA, and the difference between the optical-density at 450 nm of the cells that were treated with 0.2% dimethyl sulfoxide only and the optical density at 450 nm of the Reagent Blank wells: % control={(X−B)/(TOT−B)}×100, where X=OD450 nm of test compound well; B=OD450 of Reagent Blank wells on the ELISA; TOT=OD450 of cells that were treated with 0.2% dimethyl sulfoxide only.

Blood-Based Cytokine Production Assay

Blood was collected from normal volunteers and RA patients in heparin-containing vaccutainer tubes; these samples could be stored on ice for up to 4 hours with no adverse effect on assay performance. 75 μl of blood was placed into an individual well of a 96-well plate and diluted with 75 μl of RPMI 1640 medium containing 20 mM Hepes, pH 7.3. The diluted blood samples then were incubated for 2 hours in the absence or presence of LPS (100 ng/ml; E. coli serotype 055:B5; Sigma Chemicals; St. Louis, Mo.) at 37° C. in a 5% CO₂ environment. After this incubation, ATP was introduced as a secretion stimulus (by addition of 10 ml of a solution of 100 mM ATP in 20 mM Hepes, pH 7), and the mixtures were incubated at 37° C. for an additional 2 hours. The 96-well plates then were centrifuged at 700× g for 10 minutes, and the resulting plasma samples were harvested; these samples were stored at −20° C. Test agents to be assessed as IL-1 processing and release inhibitors were dissolved in DMSO at various concentrations and diluted into the blood samples just prior to the addition of LPS; the final concentration of DMSO vehicle in all samples was 0.2%. Each condition was assayed in a minimum of triplicate wells.

Plasma supernatants were analyzed in the following ELISAs: IL-1b (R&D Systems, Minneapolis, Minn.); IL-18 (MBL, Nagoya, Japan); TNF (R&D Systems). The assays were performed following the manufacturer's specifications, and absolute cytokine levels were calculated based on comparison to assay performance in the presence of known quantities of recombinant cytokine standards. Whole blood IC₅₀ values for the IL-1 processing and release inhibiting agents are determined from this test as the blood plasma concentration at which the absolute cytokine levels were reduced down to 50% of the levels of the controls run without any of the IL-1 processing and release inhibiting agents present.

The compounds of the present invention can be administered in a wide variety of different dosage forms, in general, the therapeutically effective compounds of this invention are present in such dosage forms at concentration levels ranging from about 5.0% to about 70% by weight. Suppositories generally contain the active ingredients in the range of 0.5% to 10% by weight; oral formulations preferably contain 10% to 70% active ingredients.

Standard methods for the procedures described in the following example, or suitable alternative procedures, are provided in widely recognized manuals of molecular biology such as, for example, Sambrook et al., Molecular Cloning, Second Edition, Cold Spring Harbor Laboratory Press (1987) and Ausabel et al., Current Protocols in Molecular Biology, Greene Publishing Associates/Wiley Interscience, New York (1990). All chemicals were either analytical grade or USP grade.

Inhibition of Human Collagenase-1 (Recombinant Collagenase-1 Assay)

This assay is used in the invention to measure the potency (IC₅₀s) of compounds for collagenase-1.

Human recombinant collagenase-1 is activated with trypsin. The amount of trypsin is optimized for each lot of collagenase-1, but a typical reaction uses the following ratio: 5 mg trypsin per 100 mg of collagenase. The trypsin and collagenase are incubated at about 20° C. to about 25° C., preferably about 23° C. for about 10 minutes then a five fold excess (50 mg/10 mg trypsin) of soybean trypsin inhibitor is added.

Stock solutions (10 mM) of inhibitors are made up in dimethylsulfoxide and then diluted using the following scheme:

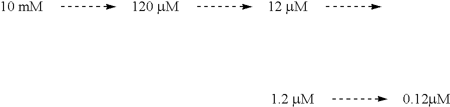

Twenty-five microliters of each concentration is then added in triplicate to appropriate wells of a 96 well microfluor plate. The final concentration of inhibitor will be a 1:4 dilution after addition of enzyme and substrate. Positive controls (enzyme, no inhibitor) are set up in wells D7-D12 and negative controls (no enzyme, no inhibitors) are set in wells D1-D6.

Collagenase-1 is diluted to 240 ng/ml and 25 ml is then added to appropriate wells of the microfluor plate. Final concentration of collagenase in the assay is 60 ng/ml.

Substrate (DNP-Pro-Cha-Gly-Cys(Me)-His-Ala-Lys(NMA)-NH₂) is made as a 5 mM stock in dimethylsulfoxide and then diluted to 20 μM in assay buffer. The assay is initiated by the addition of 50 μl substrate per well of the microfluor plate to give a final concentration of 10 μM.

Fluorescence readings (360 nM excitation, 460 nm emission) are taken at time 0 and then at about 20 minute intervals. The assay is conducted at a temperature of about 20 to about 25° C., preferably about 23° C. with a typical assay time of about 3 hours.

Fluorescence versus time is then plotted for both the blank and collagenase containing samples (data from triplicate determinations is averaged). A time point that provides a good signal (at least five fold over the blank) and that is on a linear part of the curve (usually around 120 minutes) is chosen to determine IC₅₀ values. The zero time is used as a blank for each compound at each concentration and these values are subtracted from the 120 minute data. Data is plotted as inhibitor concentration versus % control (inhibitor fluorescence divided by fluorescence of collagenase alone×100). IC₅₀s are determined from the concentration of inhibitor that gives a signal that is 50% of the control.

If IC₅₀s are reported to be less than 0.03 mM, then the inhibitors are assayed at concentrations of 0.3 μM, 0.03 μM, and 0.003 μM.

Inhibition of Human Collagenase-3 (Recombinant Collagenase-3 Assay)

This assay is used in the invention to measure the potency (IC₅₀s) of compounds for collagenase-3.

Human recombinant collagenase-3 is activated with 2mM APMA (p-aminophenyl mercuric acetate) for about 2.0 hours, at about 37° C. and is diluted to about 240 ng/ml in assay buffer (50 mM Tris, pH 7.5, 200 mM sodium chloride, 5mM calcium chloride, 20 mM zinc chloride, 0.02% BRIJ-35). Twenty-five micro-liters of diluted enzyme is added per well of a 96 well microfluor plate. The enzyme is then diluted in a 1:4 ratio by inhibitor addition and substrate to give a final concentration in the assay of 60 ng/ml.

Stock solutions (10 mM) of inhibitors are made up in dimethylsulfoxide and then diluted in assay buffer as per the inhibitor dilution scheme for inhibition of human collagenase-1: Twenty-five microliters of each concentration is added in triplicate to the microfluor plate.

The final concentrations in the assay are 30 μM, 3 μM, 0.3 μM, and 0.03 μM.

Substrate (Dnp-Pro-Cha-Gly-Cys(Me)-His-Ala-Lys(NMA)-NH₂) is prepared as for inhibition of human collagenase (collagenase-1) and 50 ml is added to each well to give a final assay concentration of 10 μM. Fluorescence readings (360 nm excitation; 450 nm emission) are taken at time 0 and about every 5 minutes for about 1 hour.

Positive controls and negative controls are set up in triplicate as outlined in the collagenase-1 assay. IC₅₀'s are determined as per inhibition of human collagenase (collagenase-1). If IC₅₀'s are reported to be less than 0.03 mM, inhibitors are then assayed at final concentrations of 0.3 μM, 0.03 μM, 0.003 μM and 0.0003 μM.

Aggrecanase Chondrocyte Assay

This assay is used in the invention to measure the potency (IC₅₀s) of compounds for aggrecanase.

Primary porcine chondrocytes from articular joint cartilage are isolated by sequential trypsin and collagenase digestion followed by collagenase digestion overnight and are plated at 2×10⁵ cells per well into 48 well plates with 5 μCi/ml³⁵S (1000 Ci/mmol) sulphur in type I collagen coated plates. Cells are allowed to incorporate label into their proteoglycan matrix (approximately 1 week) at 37° C., under an atmosphere of 5% CO₂.

The night before initiating the assay, chondrocyte monolayers are washed two times in DMEM/1% PSF/G and then allowed to incubate in fresh DMEM/1% FBS overnight.

The following morning chondrocytes are washed once in DMEM/1% PSF/G. The final wash is allowed to sit on the plates in the incubator while making dilutions. Media and dilutions can be made as described in the Table I below.

TABLE 1

| | |
|---|---|
| Control Media | DMEM alone (control media) |
| IL-1 Media | DMEM + IL-1 (5 ng/ml) |
| Drug Dilutions | Make all compounds stocks at 10 mM in DMSO. |
| | Make a 100 μM stock of each compound in DMEM in 96 |
| | well plate. Store in freezer overnight. |
| | The next day perform serial dilutions in DMEM with |
| | IL-1 to 5 μM, 500 nM, and 50 nM. |
| | Aspirate final wash from wells and add 50 μl of |
| | compound from above dilutions to 450 μl of IL-1 media |
| | in appropriate wells of the 48 well plates. |
| | Final compound concentrations equal 500 nM, 50 nM, |
| | and 5 nM. All samples completed in triplicate with |
| | Control and IL-1 alone samples on each plate. |

Plates are labeled and only the interior 24 wells of the plate are used. On one of the plates, several columns are designated as IL-1 (no drug) and Control (no IL-1, no drug). These control columns are periodically counted to monitor 35S-proteoglycan release. Control and IL-1 media are added to wells (450 μl) followed by compound (50 μl) so as to initiate the assay. Plates are incubated at 37° C., with a 5% CO₂ atmosphere.

At 40-50% release (when CPM from IL-1 media is 4-5 times control media) as assessed by liquid scintillation counting (LSC) of media samples, the assay is terminated (about 9 to about 12 hours). Media is removed from all wells and placed in scintillation tubes.

Scintillate is added and radioactive counts are acquired (LSC). To solubilize cell layers, 500 μL of papain digestion buffer (0.2 M Tris, pH 7.0, 5 mM EDTA, 5 mM DTT, and 1 mg/ml papain) is added to each well. Plates with digestion solution are incubated at 60° C. overnight. The cell layer is removed from the plates the next day and placed in scintillation tubes. 1-5 Scintillate is then added, and samples counted(LSC).

The percent of released counts from the total present in each well is determined.

Averages of the triplicates are made with control background subtracted from each well. The percent of compound inhibition is based on IL-1 samples as 0% inhibition (100% of total counts).

Inhibition of Soluble TNF-α Production (TACE Whole Blood Assay)

This assay is used in the invention to measure the potency (IC₅₀s) of compounds for TACE.

The ability of the compounds or the therapeutically acceptable salts thereof to inhibit the cellular release of TNF-α and, consequently, demonstrate their effectiveness for treating diseases involving the disregulation of soluble TNF-α is shown by the following in vitro assay:

Human mononuclear cells are isolated from anti-coagulated human blood using a one-step Ficoll-hypaque separation technique. (2) The mononuclear cells are washed three times in Hanks balanced salt solution (HBSS) with divalent cations and re-suspended to a density of 2×10⁶ /ml in HBSS containing 1% BSA. Differential counts are determined using the Abbott Cell Dyn 3500 analyzer indicated that monocytes ranged from 17 to 24% of the total cells in these preparations.

180 μL of the cell suspension was aliquoted into flat bottom 96 well plates (Costar). Additions of compounds and LPS (100 ng/ml final concentration) gives a final volume of 200 μL. All conditions are performed in triplicate. After about a four hour incubation at about 37° C. in an humidified CO₂ incubator, plates are removed and centrifuged (about 10 minutes at approximately 250× g) and the supernatants removed and assayed for TNF-α using the R&D ELISA Kit.

Note that the TACE whole blood assay, in general, gives values about 1000 fold greater than the recombinant collagenase assays. Thus, a compound with a TACE IC₅₀ of 1000 nM (i.e., 1 μM) is approximately equipotent to a collagenase IC₅₀ of 1 nM.

Inhibition of IL-18

IL-18 can be assayed according to methods analogous to those described in Wei, X., Leung, B. P., Arthur, H. M. L., McInnes, I. B., and Liew, F. Y., 2001, “Reduced incidence and severity of collagen-induced arthritis in mice lacking IL-18”, J. Immunology, 166:517-521; and Pomerantz, B. J., Reznikov, L. L., Harken, A. H., and Dinarello, C. A., 2001, “Inhibition of caspase 1 reduced human myocardial ischemic dysfunction via inhibition of IL-18 and IL-1b”, Proc. Natl. Acad. Sci., U.S.A., 98:2871-2876.

Pharmaceutical Compositions

The invention provides methods of treatment (and prophylaxis) by administration to a subject of an effective amount of a TNF inhibitor in conjunction with an IL-1/18 inhibitor (preferably an IL-1 processing and release inhibiting agent). The subject is preferably an animal, including but not limited to animals such as cows, pigs, chickens, primates, etc., and is preferably a mammal, and most preferably human.

Because it is possible that the inhibitory function of the preferred inhibitors is imparted by one or more discrete and separable portions, it is also envisioned that the methods of the present invention can be practiced by administering a therapeutic composition having as an active ingredient a portion or portions of the TNF inhibitor or IL-1/18 inhibitor that control(s) interleukin-1/18 or TNF inhibition.

The therapeutic composition of the present invention can be administered parenterally by injection, although other effective administration forms, such as intraarticular injection, inhalant mists, orally active formulations, transdermal iontophoresis or suppositories, are also envisioned. One preferred carrier is physiological saline solution, but it is contemplated that other pharmaceutically acceptable carriers may also be used.

In one embodiment, it is envisioned that the carrier and the TNF inhibitor and the IL-1/18 inhibitor constitute a physiologically-compatible, slow-release formulation. The primary solvent in such a carrier can be either aqueous or non-aqueous in nature. In addition, the carrier can contain other pharmacologically-acceptable excipients for modifying or maintaining the pH, osmolarity, viscosity, clarity, color, sterility, stability, rate of dissolution, or odor of the formulation. Similarly, the carrier can contain still other pharmacologically-acceptable excipients for modifying or maintaining the stability, rate of dissolution, release, or absorption of the TNF inhibitor and/or IL-1/18 inhibitor. Such excipients are those substances usually and customarily employed to formulate dosages for parenteral administration in either unit dose or multi-dose form.

Once the therapeutic composition has been formulated, it can be stored in sterile vials as a solution, suspension, gel, emulsion, solid, or dehydrated or lyophilized powder. Such formulations may be stored either in a ready to use form or requiring reconstitution immediately prior to administration. The preferred storage of such formulations is at temperatures at least as low as 4° C. and preferably at −70° C. It is also preferred that such formulations containing a TNF inhibitor and a IL-1/18 inhibitor are stored and administered at or near physiological pH. It is presently believed that administration in a formulation at a high pH (i.e. greater than 8) or at a low pH (i.e. less than 5) is undesirable.

Preferably, the manner of administering the formulations containing the TNF inhibitor and the IL-1/18 inhibitor for systemic delivery is via subcutaneous, intramuscular, intravenous, intranasal, or vaginal or rectal suppository. Preferably the manner of administration of the formulations containing a TNF inhibitor and an IL-1/18 inhibitor for local delivery is via intraarticular, intratracheal, or instillation or inhalations to the respiratory tract. In addition it may be desirable to administer the TNF inhibitor and IL-1/18 inhibitor to specified portions of the alimentary canal either by oral administration of the TNF inhibitor and the IL-1/18 inhibitor in an appropriate formulation or device or by suppository or enema.

In an additional preferred mode for the treatment of TNF and IL-1/18 mediated diseases an initial intravenous bolus injection of TNF inhibitor and IL-1/18 inhibitor is administered followed by a continuous intravenous infusion of TNF inhibitor and IL-1/18 inhibitor. The initiation of treatment for septic shock should be begun as soon as possible after septicemia or the chance of septicemia is diagnosed. For example, treatment may be begun immediately following surgery or an accident or any other event that may carry the risk of initiating septic shock.

Preferred modes for the treatment of TNF or IL-1/18 mediated diseases and more particularly for the treatment of arthritis include: (1) a single intraarticular injection of TNF inhibitor and IL-1/18 inhibitor given periodically as needed to prevent or remedy flare up of arthritis; and (2) periodic subcutaneous injections of TNF inhibitor and IL-1/18 inhibitor.

Preferred modes for the treatment of TNF and ]IL-1/18 mediated diseases and more particularly for the treatment of adult respiratory distress syndrome include: 1) single or multiple intratracheal administrations of TNF inhibitor and IL-1/18 inhibitor-, and 2) bolus or continuous intravenous infusion of TNF inhibitor and IL-1/18 inhibitor.

It is also contemplated that certain formulations containing TNF inhibitor and IL-1/18 inhibitor are to be administered orally. Preferably, when the TNF inhibitor and IL-1/18 inhibitor is a protein, the administration in this fashion is encapsulated. The encapsulated TNF inhibitor and/or IL-1/18 inhibitor may be formulated with or without those carriers customarily used in the compounding of solid dosage forms. Preferably, the capsule is designed so that the active portion of the formulation is released at that point in the gastrointestinal tract when bioavailability is maximized and pre-systemic degradation is minimized. Additional excipients may be included to facilitate absorption of the TNF inhibitor and IL-1/18 inhibitor. Diluents, flavorings, low melting point waxes, vegetable oils, lubricants, suspending agents, tablet disintegrating agents, and binders may also be employed.

For oral administration when the TNF inhibitor and IL-1/18 inhibitor are non-peptidic (e.g., an IL-1 processing and release inhibitor, an ICE inhibitor or a TACE inhibitor), tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelation and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

Administration can also be systemic or local. In addition, it may be desirable to introduce a TNF inhibitor in conjunction with an agent inhibiting the propagation of IL-1/18 into the inflammed joint by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the TNF inhibitor in conjunction with an agent inhibiting the propagation of IL-1/18 locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Thus, one preferred embodiment of the invention provides a pharmaceutical composition comprising a combination of a TNF inhibitor with an IL-1 processing and release inhibiting agent or an IL-1ra, and one or more ingredients selected from the group consisting of a pharmaceutically acceptable carrier, a pharmaceutically acceptable excipient, a wetting agent, a buffering agent, an emulsifying agent, and a binding agent.

In another preferred embodiment, a kit is provided comprising in one or more containers a combination of a TNF inhibitor with an IL-1 processing and release inhibiting agent or an IL-1ra.

The dosage range required depends on the choice of TNF inhibitor and the agent inhibiting the propagation of IL-1/18, the route of administration, the nature of the formulation, the nature of the subject's condition, and the judgment of the attending practitioner.

In certain embodiments, the administration is designed to create a preselected concentration range of TNF inhibitor and IL-1/18 inhibitor in the patients blood stream. it is believed that the maintenance of circulating concentrations of TNF inhibitor and IL-1/18 inhibitor of less than 0.01 ng per ml of plasma may not be an effective composition, while the prolonged maintenance of circulating levels in excess of 10 μg per ml may have undesirable side, effects.

Further refinement of the calculations necessary to determine the appropriate dosage for treatment involving each of the above mentioned formulations is routinely made by those of ordinary skill in the art and is within the skill routinely performed by them without undue experimentation, especially in light of the dosage information and assays disclosed herein. These dosages may be ascertained through use of the established assays for determining dosages utilized in conjunction with appropriate dose-response data.

Suitable once or twice twice-daily dosages for the TNF inhibitor, however, are in the range of 1-1000 μg/kg of subject in combination with 50-1200 mg of an agent inhibiting the propagation of IL-1/18. Wide variations in the needed dosage, however, are to be expected in view of the variety of compounds available and the differing efficiencies of various routes of administration.

Oral administration would be expected to require higher dosages than administration by intravenous injection. Variations in these dosage levels can be made using standard empirical routines for optimization, as is well understood in the art.

Compositions comprising TNF inhibitor and an agent inhibiting the propagation of IL-1/18 can be administered in a wide variety of dosage forms. In general, the therapeutically effective compounds of this invention are present in such dosage forms at concentration levels ranging from about 5.0% to about 70% by weight.

It should be noted that the TNF inhibitor and IL-1/18 inhibitor formulations described herein may be used for veterinary as well as human applications and that the term “patient” should not be construed in a limiting manner. In the case of veterinary applications, the dosage ranges should be the same as specified above.

The TNF inhibitor in conjunction with an agent inhibiting the propagation of IL-1/18 may be administered together with other biologically active agents. Preferred biologically active agents for administration in combination with the TNF inhibitor and an agent inhibiting the propagation of IL-1/18 are NSAIDs, especially COX-2 selective inhibitors (e.g. celecoxib, valdecoxib, rofecoxib and etoricoxib), and matrix metalloproteases.

The foregoing description of the invention is exemplary for purposes of illustration and explanation. It will be apparent to those skilled in the art that changes and modifications are possible without departing from the spirit and scope of the invention. It is intended that the following claims be interpreted to embrace all such changes and modifications.

1. A composition for treating inflammation comprising an amount of an IL-1 inhibitor in combination with an amount of a Tumor Necrosis Factor (TNF) inhibitor, wherein the amount of the two components is effective for treating inflammation and a pharmaceutically acceptable carrier.

2. A composition for treating inflammation comprising an amount of an IL-1 and an IL-18 inhibitor in combination with an amount of a Tumor Necrosis Factor (TNF) inhibitor, wherein the amount of the two components is effective for treating inflammation and a pharmaceutically acceptable carrier.

3. The composition according to claim 1, wherein said IL-1 inhibitor is selected from the group consisting of an IL-1 processing and release inhibitor.

4. The composition according to claim 1, wherein said IL-1 inhibitor is an IL-1 processing and release inhibitor selected from the group consisting of an ICE inhibitor, a caspase inhibitor, and an IL-1 post-translational processing inhibitor.

5. The composition according to claim 1, wherein said IL-1 inhibitor is an ICE inhibitor.

6. The composition according to claim 1, wherein said IL-1 inhibitor is a caspase inhibitor.

7. The composition according to claim 1, wherein said IL-1 inhibitor is an IL-1 post-translational processing inhibitor.

8. The composition according to claim 1, wherein said IL-1 inhibitor is a diarylsulfonylurea.

9. The composition according to claim 8, wherein said diarylsulfonylurea is selected from the group consisting of
1-(1,2,3,5,6,7-Hexahydro-s-indacen-4-yl)-3-[4-(1-hydroxy-1-methyl-ethyl)-furan-2-sulfonyl]-urea;
1-(2,6-Diisopropyl-phenyl)-3-[4-(1-hydroxy-1-methyl-ethyl)-furan-2-sulfonyl]-urea;
4-Chloro-2,6-diisopropyl-phenyl-3-[4-(1-hydroxy-1-methyl-ethyl)-furan-2-sulfonyl]-urea;
1,2,3,5,6,7-Hexahydro-4-aza-s-indacen-8-yl-3-[4-(1-hydroxy-1-methyl-ethyl)-furan-2-sulfonyl]-urea;
8-Chloro-1,2,3,5,6,7-hexahydro-s-indacen-4-yl-3-[4-(1-hydroxy-1-methyl-ethyl)-furan-2-sulfonyl]-urea;
8-Fluoro-1,2,3,5,6,7-hexahydro-s-indacen-4-yl-3-[4-(1-hydroxy-1-methyl-ethyl)-furan-2-sulfonyl]-urea; and
4-Fluoro-2,6-diisopropyl-phenyl-3-[4-(1-hydroxy-1-methyl-ethyl)-furan-2-sulfonyl]-urea.

10. The composition according to claim 1, wherein said Tumor Necrosis Factor (TNF) inhibitor is etanercept.

11. The composition according to claim 1, wherein said Tumor Necrosis Factor (TNF) inhibitor is infliximab.

12. The composition according to claim 1, wherein said Tumor Necrosis Factor (TNF) inhibitor is CDP-870.

13. The composition according to claim 1, wherein said Tumor Necrosis Factor (TNF) inhibitor is adalimumab.

14. The composition according to claim 1, wherein said Tumor Necrosis Factor (TNF) inhibitor is a TACE inhibitor.

15. The composition according to claim 1, wherein said Tumor Necrosis Factor (TNF) inhibitor is an ADAM-17 inhibitor 100 fold selective for ADAM-17 over each of MMP-1, 2, 3, 4, 5, 6, 7, 8, 9, 10, 11, 12 and 14 as defined in an in vitro assay.

16. The composition according to claim 15, wherein said IL-1 inhibitor is an IL-1ra.

17. The composition according to claim 15, wherein said IL-1 inhibitor is the IL-1ra anakinra.

18. The composition according to claim 1, wherein said a Tumor Necrosis Factor (TNF) inhibitor is an arylsulfonyl hydroxamic acid derivative.

19. A method of treating inflammation comprising administering to a mammal in need thereof an amount of an IL-1 inhibitor in combination with an amount of a Tumor Necrosis Factor (TNF) inhibitor, wherein the amount of the two components is effective for treating inflammation.

20. A method of treating inflammation comprising administering to a mammal in need thereof an amount of an IL-1 inhibitor and an IL-18 inhibitor in combination with an amount of a Tumor Necrosis Factor (TNF) inhibitor, wherein the amount of the two components is effective for treating inflammation.

21. The method according to claim 19, wherein said IL-1 inhibitor is selected from the group consisting of an IL-1 processing and release inhibitor.

22. The method according to claim 19, wherein said IL-1 inhibitor is an IL-1 processing and release inhibitor selected from the group consisting of an ICE inhibitor and an IL-1 post-translational processing inhibitor.

23. The method according to claim 19, wherein said IL-1 inhibitor is an ICE inhibitor.

24. The method according to claim 19, wherein said IL-1 inhibitor is the ICE inhibitor VX740.

25. The method according to claim 19, wherein said IL-1 inhibitor is an IL-1 post-translational processing inhibitor.

26. The method according to claim 19, wherein said IL-1 inhibitor is a diarylsulfonylurea.

27. The method according to claim 19, wherein said diarylsulfonylurea is selected from the group consisting of
1-(1,2,3,5,6,7-Hexahydro-s-indacen-4-yl)-3-[4-(1-hydroxy-1-methyl-ethyl)-furan-2-sulfonyl]-urea;
1-(2,6-Diisopropyl-phenyl)-3-[4-(1-hydroxy-1-methyl-ethyl)-furan-2-sulfonyl]-urea;
4-Chloro-2,6-diisopropyl-phenyl-3-[4-(1-hydroxy-1-methyl-ethyl)-furan-2-sulfonyl]-urea;
1,2,3,5,6,7-Hexahydro-4-aza-s-indacen-8-yl-3-[4-(1-hydroxy-1-methyl-ethyl)-furan-2-sulfonyl]-urea;
8-Chloro-1,2,3,5,6,7-hexahydro-s-indacen-4-yl-3-[4-(1-hydroxy-1-methyl-ethyl)-furan-2-sulfonyl]-urea;
8-Fluoro-1,2,3,5,6,7-hexahydro-s-indacen-4-yl-3-[4-(1-hydroxy-1-methyl-ethyl)-furan-2-sulfonyl]-urea; and
4-Fluoro-2,6-diisopropyl-phenyl-3-[4-(1-hydroxy-1-methyl-ethyl)-furan-2-sulfonyl]-urea.

28. The method according to claim 19, wherein said Tumor Necrosis Factor (TNF) inhibitor is etanercept.

29. The method according to claim 19, wherein said Tumor Necrosis Factor (TNF) inhibitor is infliximab.

30. The method according to claim 19, wherein said Tumor Necrosis Factor (TNF) inhibitor is CDP-870.

31. The method according to claim 19, wherein said Tumor Necrosis Factor (TNF) inhibitor is adalimumab.

32. The method according to claim 19, wherein said Tumor Necrosis Factor (TNF) inhibitor is a TACE inhibitor.

33. The method according to claim 19, wherein said Tumor Necrosis Factor (TNF) inhibitor is an ADAM-17 inhibitor 100 fold selective for ADAM-17 over each of MMP-1, 2, 3, 4, 5, 6, 7, 8, 9, 10, 11, 12 and 14 as each are defined in in vitro assays.

34. The method according to claim 33, wherein said IL-1 inhibitor is an IL-1ra.

35. The method according to claim 33, wherein said IL-1 inhibitor is the IL-1ra anakinra.

36. The method according to claim 19, wherein said a Tumor Necrosis Factor (TNF) inhibitor is an arylsulfonyl hydroxamic acid derivative.
